SEQUENCE LISTING (20 sequences)

SEQ ID NO: 1 - DNA, 2219 bp, Homo sapiens
aattccggagccatggtgaacgaagccagaggaaacagcagcctcaacccctgcttggagggcagtgccagcagtggcagtgagagctccaaagatagttcgagatgttccaccccgggcctggaccctgagcggcatgagagactccgggagaagatgaggcggcgattggaatctggtgacaagtggttctccctggaattcttccctcctcgaactgctgagggagctgtcaatctcatctcaaggtttgaccggatggcagcaggtggccccctctacatagacgtgacctggcacccagcaggtgaccctggctcagacaaggagacctcctccatgatgatcgccagcaccgccgtgaactactgtggcctggagaccatcctgcacatgacctgctgccgtcagcgcctggaggagatcacgggccatctgcacaaagctaagcagctgggcctgaagaacatcatggcgctgcggggagacccaataggtgaccagtgggaagaggaggagggaggcttcaactacgcagtggacctggtgaagcacatccgaagtgagtttggtgactactttgacatctgtgtggcaggttaccccaaaggccaccccgaagcagggagctttgaggctgacctgaagcacttgaaggagaaggtgtctgcgggagccgatttcatcatcacgcagcttttctttgaggctgacacattcttccgctttgtgaaggcatgcaccgacatgggcatcacttgccccatcgtccccgggatctttcccatccagggctaccactcccttcggcagcttgtgaagctgtccaagctggaggtgccacaggagatcaaggacgtgattgagccaatcaaagacaacgatgctgccatccgcaactatggcatcgagctggccgtgagcctgtgccaggagcttctggccagtggcttggtgccaggcctccacttctacaccctcaaccgcgagatggctaccacagaggtgctgaagcgcctggggatgtggactgaggaccccaggcgtcccctaccctgggctctcagtgcccaccccaagcgccgagaggaagatgtacgtcccatcttctgggcctccagaccaaagagttacatctaccgtacccaggagtgggacgagttccctaacggccgctggggcaattcctcttcccctgcctttggggagctgaaggactactacctcttctacctgaagagcaagtcccccaaggaggagctgctgaagatgtggggggaggagctgaccagtgaagcaagtgtctttgaagtctttgttctttacctctcgggagaaccaaaccggaatggtcacaaagtgacttgcctgccctggaacgatgagcccctggcggctgagaccagcctgctgaaggaggagctgctgcgggtgaaccgccagggcatcctcaccatcaactcacagcccaacatcaacgggaagccgtcctccgaccccatcgtgggctggggccccagcgggggctatgtcttccagaaggcctacttagagtttttcacttcccgcgagacagcggaagcacttctgcaagtgctgaagaagtacgagctccgggttaattaccaccttgtcaatgtgaagggtgaaaacatcaccaatgcccctgaactgcagccgaatgctgtcacttggggcatcttccctgggcgagagatcatccagcccaccgtagtggatcccgtcagcttcatgttctggaaggacgaggcctttgccctgtggattgagcggtggggaaagctgtatgaggaggagtccccgtcccgcaccatcatccagtacatccacgacaactacttcctggtcaacctggtggacaatgacttcccactggacaactgcctctggcaggtggtggaagacacattggagcttctcaacaggcccacccagaatgcgagagaaacggaggctccatgaccctgcgtcctgacgccctgcgttggagccactcctgtcccgccttcctcctccacagtgctgcttctcttgggaactccactctccttcgtgtctctcccaccccggcctccactcccccacctgacaatggcagctagactggagtgaggcttccaggctcttcctggacctgagtcggccccacatgggaacctagtactctctgctctaaaaaaaaaaaaaaaaaaggaatt

SEQ ID NO: 2 - protein, 656 aa, Homo sapiens
MVNEARGNSSLNPCLEGSASSGSESSKDSSRCSTPGLDPERHERLREKMRRRLESGDKWFSLEFFPPRTAEGAVNLISRFDRMAAGGPLYIDVTWHPAGDPGSDKETSSMMIASTAVNYCGLETILHMTCCRQRLEEITGHLHKAKQLGLKNIMALRGDPIGDQWEEEEGGFNYAVDLVKHIRSEFGDYFDICVAGYPKGHPEAGSFEADLKHLKEKVSAGADFIITQLFFEADTFFRFVKACTDMGITCPIVPGIFPIQGYHSLRQLVKLSKLEVPQEIKDVIEPIKDNDAAIRNYGIELAVSLCQELLASGLVPGLHFYTLNREMATTEVLKRLGMWTEDPRRPLPWALSAHPKRREEDVRPIFWASRPKSYIYRTQEWDEFPNGRWGNSSSPAFGELKDYYLFYLKSKSPKEELLKMWGEELTSEASVFEVFVLYLSGEPNRNGHKVTCLPWNDEPLAAETSLLKEELLRVNRQGILTINSQPNINGKPSSDPIVGWGPSGGYVFQKAYLEFFTSRETAEALLQVLKKYELRVNYHLVNVKGENITNAPELQPNAVTWGIFPGREIIQPTVVDPVSFMFWKDEAFALWIERWGKLYEEESPSRTIIQYIHDNYFLVNLVDNDFPLDNCLWQVVEDTLELLNRPTQNARETEAP

SEQ ID NO: 3 - DNA, 346 bp, Homo sapiens
  misc_feature (326)...(328): n= a, t, g or c
gggtgtggctgcctgccccctgatgctccctgccccaccctgtgcagtaggaacccagccatggtgaacgaagccagaggaaacagcagcctcaacccctgcttggagggcagtgccagcagtggcagtgagagctccaaagatagttcgagatgttccaccccgggcctggaccctgagcggcatgagagactccgggagaagatgaggcggcgattggaatctggtgacaagtggttctccctggaattcttccctcctcgaactgctgagggagctgtcaatctcatctcaaggtaaactcatgcaaggttaaggtgggaggnnngagtggtggtgcctgggg

SEQ ID NO: 4 - DNA, 339 bp, Homo sapiens
acggatggtatttctcctggaacctctcttcagaaacaaaccccctacaggtttgaccggatggcagcaggtggccccctctacatagacgtgacctggcacccagcaggtgaccctggctcagacaaggagacctcctccatgatgatcgccagcaccgccgtgaactactgtggcctggagaccatcctgcacatgacctgctgccgtcagcgcctggaggagatcacgggccatctgcacaaagctaagcagctgggcctgaagaacatcatggcgctgcggggaggtgtggagccagcactcccctacactctgggttctggctttcccggaggc

SEQ ID NO: 5 - DNA, 210 bp, Homo sapiens
tctggaggttgggtgagacccagtgactatgacctccaccaaccctgcagacccaataggtgaccagtgggaagaggaggagggaggcttcaactacgcagtggacctggtgaagcacatccgaagtgagtttggtgactactttgacatctgtgtggcaggtgagtggctggatcatcctggtggcggggatggagctagggaggctga

SEQ ID NO: 6 - DNA, 294 bp, Homo sapiens
  misc_feature (260): n = a, t, g or c
ccttgaacaggtggaggccagcctctcctgactgtcatccctattggcaggttaccccaaaggccaccccgaagcagggagctttgaggctgacctgaagcacttgaaggagaaggtgtctgcgggagccgatttcatcatcacgcagcttttctttgaggctgacacattcttccgctttgtgaaggcatgcaccgacatgggcatcacttgccccatcgtccccgggatctttcccatccaggtgaggggcccagganagcccataagctccctccaccccactctcaccgc

SEQ ID NO: 7 - DNA, 341 bp, Homo sapiens
gctggccagcagccgccacagcccctcatgtcttggacagggctaccactcccttcggcagcttgtgaagctgtccaagctggaggtgccacaggagatcaaggacgtgattgagccaatcaaagacaacgatgctgccatccgcaactatggcatcgagctggccgtgagcctgtgccaggagcttctggccagtggcttggtgccaggcctccacttctacaccctcaaccgcgagatggctaccacagaggtgctgaagcgcctggggatgtggactgaggaccccaggtgagggcagtggcccagagatccccagaggagggtccaagagcagcccc

SEQ ID NO: 8 - DNA, 235 bp, Homo sapiens
tccctctagccaatcccttgtctcaattctctgtccccatcctcacccaggcgtcccctaccctgggctctcagtgcccaccccaagcgccgagaggaagatgtacgtcccatcttctgggcctccagaccaaagagttacatctaccgtacccaggagtgggacgagttccctaacggccgctggtgagggcctgcagaccttccttgcaaatacatctttgttcttgggagcg

SEQ ID NO: 9 - DNA, 281 bp, Homo sapiens
actgccctctgtcaggagtgtgccctgacctctgggcacccctctgccaggggcaattcctcttcccctgcctttggggagctgaaggactactacctcttctacctgaagagcaagtcccccaaggaggagctgctgaagatgtggggggaggagctgaccagtgaagcaagtgtctttgaagtctttgttctttacctctcgggagaaccaaaccggaatggtcacaaagtgagtgatgctggaagtggggaccctggttcatcccctgcccctggcct

SEQ ID NO: 10 - DNA, 283 bp, Homo sapiens
cagggtgccaaacctgatggtcgccccagccagctcaccgtctctcccaggtgacttgcctgccctggaacgatgagcccctggcggctgagaccagcctgctgaaggaggagctgctgcgggtgaaccgccagggcatcctcaccatcaactcacagcccaacatcaacgggaagccgtcctccgaccccatcgtgggctggggccccagcgggggctatgtcttccagaaggtgtggtagggaggcacggggtgcccccctctcttgaccggcacccgtgg

SEQ ID NO: 11 - DNA, 202 bp, Homo sapiens
  misc_feature (187): n = a, t, g or c
gggcgtctggcagggctggggttggtgacaggcacctgtctctcccacaggcctacttagagtttttcacttcccgcgagacagcggaagcacttctgcaagtgctgaagaagtacgagctccgggttaattaccaccttgtcaatgtgaaggtaggccaggccccacggttcccacagagtaccangcccttcnttgaaca

SEQ ID NO: 12 - DNA, 220 bp, Homo sapiens
actccagttgttcttggcccaggtcttacccccaccccacatcccctcagggtgaaaacatcaccaatgcccctgaactgcagccgaatgctgtcacttggggcatcttccctgggcgagagatcatccagcccaccgtagtggatcccgtcagcttcatgttctggaaggtaaaggagcgggggcaagcttgccccgcccacctggaaaaccgtgggga

SEQ ID NO: 13 - DNA, 522 bp, Homo sapiens
  misc_feature (512): n = a, t, g or c
ctctgtgtgtgtgtgcatgtgtgcgtgtgtgcgggggtatgtgtgtgtaggacgaggcctttgccctgtggattgagcggtggggaaagctgtatgaggaggagtccccgtcccgcaccatcatccagtacatccacgacaactacttcctggtcaacctggtggacaatgacttcccactggacaactgcctctggcaggtggtggaagacacattggagcttctcaacaggcccacccagaatgcgagagaaacggaggctccatgaccctgcgtcctgacgccctgcgttggagccactcctgtcccgccttcctcctccacagtgctgcttctcttgggaactccactctccttcgtgtctctcccaccccggcctccactcccccacctgacaatggcagctagactggagtgaggcttccaggctcttcctggacctgagtcggccccacatgggaacctagtactctctgctctagccaggagtctgtgctcttttggtggggancacttgcntc

SEQ ID NO: 14 - DNA, 20 bp, Artificial Sequence
  Synthetic Primer
agctgccgaagggagtggta

SEQ ID NO: 15 - DNA, 20 bp, Artificial Sequence
  Antisense Oligonucleotide
gtgacgtaggacagcgatgg

SEQ ID NO: 16 - DNA, 20 bp, Artificial Sequence
  Antisense Oligonucleotide
tgctgtcggagcgataggtc

SEQ ID NO: 17 - DNA, 20 bp, Artificial Sequence
  Control Antisense Oligonucleotide
gtgacgtaggacagcgatgg

SEQ ID NO: 18 - DNA, 20 bp, Artificial Sequence
  Control Antisense Oligonucleotide
tgctgtcggagcgataggtc

SEQ ID NO: 19 - DNA, 20 bp, Artificial Sequence
  Antisense Oligonucleotide
agctgccgaagggagtggta

SEQ ID NO: 20 - DNA, 20 bp, Artificial Sequence
  Control Antisense Oligonucleotide
gtggactaggacagcgatgg

(19) United States
(12) Patent Application Publication
(10) Pub. No.: US 2005/0222063 A1
(43) Pub. Date: Oct. 6, 2005

(54) Methylenetetrahydrofolate reductase inhibitors and use thereof

(76) Inventors: Rima Rozen, Montreal West (CA); Jaspreet Sekhon, Vancouver (CA)

Correspondence Address: Albert Wai-Kit Chan;Law Offices of Albert Wai-Kit Chan LLC, World Plaza, Suite 604, 141-07 20th Avenue, Whitestone, NY, 11357, US

(21) Appl. No.: 10/831,759

(22) Filed: Apr. 23, 2004

Related U.S. Application Data
(63) Continuation-in-part of application No. 10/433,392, filed on Nov. 20, 2003.

(51) Int. Cl. A61K 48/00; C07H 21/02

(52) U.S. Cl. 514/44; 536/23.1

(57) ABSTRACT

The present invention provides methylenetetrahydrofolate reductase (MTHFR) inhibitors, such as antisense oligonucleotides, for use in selective inhibition of cancer cell growth in a mammal. The present invention further provides methods of using the MTHFR inhibitors, alone or in combination with one or more standard chemotherapeutics, for selective inhibition of cancer cell growth.

### CROSS REFERENCES TO RELATED APPLICATION

This application is a continuation-in-part of U.S. patent application Ser. No. 10/433,392, filed Jun. 2, 2003, which is a national stage of PCT/CA01/01697, filed Dec. 3, 2001. The aforesaid PCT application claims priority from U.S. Provisional Patent Application Ser. No. 09/728,910, filed Dec. 1, 2000. The contents of all of the aforementioned applications are hereby specifically incorporated by reference in their entirety.

### FIELD OF THE INVENTION

The present invention pertains to the field of inhibitors of cancer cell growth and/or metastasis. More particularly the present invention pertains to inhibitors of methylenetetrahydrofolate reductase and use thereof in the treatment of cancer.

### BACKGROUND

Folic acid derivatives are coenzymes for several critical single-carbon transfer reactions, including reactions in the biosynthesis of purines, thymidylate and methionine. Methylenetetrahydrofolate reductase (MTHFR; EC 1.5.1.20) catalyses the NADPH-linked reduction of 5,10-methylenetetrahydrofolate to 5-methyltetrahydrofolate, a co-substrate for methylation of homocysteine to methionine.

Hereditary deficiency of MTHFR, an autosomal recessive disorder, is the most common inborn error of folic acid metabolism. A block in the production of methyltetrahydrofolate leads to elevated homocysteine with low to normal levels of methionine. Patients with severe deficiencies of MTHFR (0-20% activity in fibroblasts) can have variable phenotypes. Developmental delay, mental retardation, motor and gait abnormalities, peripheral neuropathy, seizures and psychiatric disturbances have been reported in this group, although at least one patient with severe MTHFR deficiency was asymptomatic. Pathologic changes in the severe form include the vascular changes that have been found in other conditions with elevated homocysteine, as well as reduced neurotransmitter and methionine levels in the CNS. A milder deficiency of MTHFR (35-50% activity) has been described in patients with coronary artery disease (see below). Genetic heterogeneity is likely, considering the diverse clinical features, the variable levels of enzyme activity, and the differential heat inactivation profiles of the reductase in patients' cells.

MTHFR isolated from porcine liver has been purified to homogeneity and has been found to be a homodimer of 77-kDa subunits. Partial proteolysis of the porcine peptide has revealed two spatially distinct domains: an N-terminal domain of 40 kDa and a C-terminal domain of 37 kDa. The latter domain contains the binding site for the allosteric regulator S-adenosylmethionine.

The cDNA for human MTHFR has been isolated and mapped, and mutations in the gene have been identified in MTHFR-deficient individuals (Goyette, et al., (1994) Nat. Genet., 7:195-200). International Patent Application No. PCT/IB00/00442 discloses nucleic acid probes for the MTHFR gene, methods of identifying mutations in the MTHFR gene of individuals with MTHFR deficiency and methods of treatment for individuals with MTHFR deficiency involving the provision of a functional MTHFR gene or protein. The application further teaches that the MTHFR deficiency may be associated with a disease, disorder or dysfunction including cancers such as neuroblastomas and colorectal carcinomas.

PCT/IB00/00442 also postulates about a method for treating a patient having a cancer by inhibiting MTHFR gene expression or by inhibiting the MTHFR protein. However, given the teaching therein, it remains uncertain whether such a method would be effective in the treatment of cancer especially in view of the demonstrated link between MTHFR deficiency and disease. PCT/IB00/00442 does not discuss how the treatment of a patient having a cancer by inhibiting MTHFR gene expression or the MTHFR protein could be implemented or what effect such inhibition may have on cancer cells. In fact, PCT/IB00/00442 appears to teach that reducing MTHFR activity in a subject will have a deleterious effect.

There remains, therefore, a need for a method of selectively targeting cancer cells. In particular for a method that provides specific inhibition of the growth of cancer cells.

This background information is provided for the purpose of making known information believed by the applicant to be of possible relevance to the present invention. No admission is necessarily intended, nor should be construed, that any of the preceding information constitutes prior art against the present invention. Publications referred to throughout the specification are hereby incorporated by reference in their entireties in this application.

### SUMMARY OF THE INVENTION

An object of the present invention is to provide methylenetetrahydrofolate reductase inhibitors and use thereof. In accordance with an aspect of the present invention, there is provided an oligonucleotide inhibitor of methylenetetrahydrofolate reductase (MTHFR) between about 7 and about 100 nucleotides in length comprising a sequence that is complementary to a human MTHFR mRNA, wherein the oligonucleotide inhibits human MTHFR gene expression.

In accordance with another aspect of the invention, there is provided a vector comprising a nucleic acid encoding an oligonucleotide inhibitor of the invention.

In accordance with another aspect of the invention, there is provided a method of treating, stabilizing or preventing cancer in a mammal comprising administering to said mammal an oligonucleotide inhibitor of methylenetetrahydrofolate reductase (MTHFR) between about 7 and about 100 nucleotides in length comprising a sequence that is complementary to a human MTHFR mRNA, wherein the oligonucleotide inhibits human MTHFR gene expression.

In accordance with another aspect of the invention, there is provided a method of treating, stabilizing or preventing cancer in a mammal comprising administering to said mammal an oligonucleotide inhibitor of methylenetetrahydrofolate reductase (MTHFR) in combination with one or more chemotherapeutic, wherein the oligonucleotide is between about 7 and about 100 nucleotides in length and comprises a sequence that is complementary to a human MTHFR mRNA, and wherein the oligonucleotide inhibits human MTHFR gene expression.

In accordance with another aspect of the invention, there is provided a method of inhibiting growth of cancer cells comprising the step of contacting said cancer cells with an oligonucleotide inhibitor of methylenetetrahydrofolate reductase (MTHFR) between about 7 and about 100 nucleotides in length comprising a sequence that is complementary to a human MTHFR mRNA, wherein the oligonucleotide inhibits human MTHFR gene expression.

### BRIEF DESCRIPTION OF THE FIGURES

FIG. 1 depicts the total available sequence (SEQ ID NO:1 and NO:2) of human MTHFR cDNA.

FIG. 2 depicts human MTHFR exons and flanking intronic sequences. The exonic sequences (SEQ ID NOs: 3-13) of the human gene are given, along with their sizes and flanking intronic sequences. The base pair location of the exons within the cDNA, given in parenthesis, relates to the published human cDNA base pair numbering (Goyette et al., 1994). Base 1 is 12 bp upstream from the ATG in original cDNA, the equivalent base is indicated here by an asterisk. Exon 1 contains the ATG start site (underlined), and exon 11 contains the termination codon (underlined).

FIG. 3 demonstrates the percent survival of SW620 colon carcinoma cells following three rounds of treatment for five hours each round with varying concentrations of an antisense phosphorothioate oligonucleotide to exon 5 of MTHFR. The cells were allowed to recover after the final treatment for a period of two days before the cells were counted. The values are expressed as the percent of cells surviving compared to the number of cells which survived after treatment with a control oligonucleotide CTSEX5 (phosphorothioate 5′-GTGACGTAGGACAGCGATGG-3′; SEQ ID NO:17).

FIG. 4 demonstrates the percent survival of LOVO colon carcinoma cells treated with an MTHFR antisense oligonucleotide after a recovery period of three days, as described for FIG. 3.

FIG. 5 demonstrates the percent survival of BEC 2 neuroblastoma cells treated with varying concentrations of an MTHFR antisense oligonucleotide and allowed to recover for two days, as described for FIG. 3.

FIG. 6 demonstrates the percent survival of SK-N-F1 neuroblastoma cells treated with varying concentrations of an MTHFR antisense oligonucleotide after a recovery period of four days, as described for FIG. 3.

FIG. 7 demonstrates the percent survival of MCF7 breast cancer cells that were treated with different concentrations of an MTHFR antisense oligonucleotide and allowed to recover for 2.5 days, as described for FIG. 3.

FIG. 8 demonstrates the percent survival of SKBr3 breast cancer cells treated with varying concentrations of an MTHFR antisense oligonucleotide after a recovery period of 2.5 days, as described for FIG. 3.

FIG. 9 demonstrates the percent survival of U87-lacZ glioma cells treated with varying concentrations of an MTHFR antisense oligonucleotide after a recovery period of two days, as described for FIG. 3. For this experiment an oligonucleotide with six base pair mismatches CT677 (phosphorothioate 5′-TGCTGTCGGAGCGATAGGTC-3′; SEQ ID NO:18) was used as the control oligonucleotide.

FIG. 10 demonstrates the percent survival of WG1554 fibroblast cells homozygous for a nonsense mutation in MTHFR which were treated with 400 nM MTHFR antisense or control oligonucleotide and allowed to recover for three days, as described for FIG. 3.

FIG. 11 depicts the growth of fibroblast cell lines in deficient media. Two wild type fibroblast cell lines (MCH 51, MCH 75) and an MTHFR null mutant (WG 1554) were grown in MEM (▪), M- (X), and M-H+ (μ) for 12 days. The number of cells for each line was counted using the SRB assay at 3 time points. Each point represents the mean of 3 replicates ±SD.

FIG. 12 depicts the growth of colon carcinoma cell lines in deficient media. Four colon carcinoma cell lines were grown in MEM (▪), M- (X), and M-H+ (μ) for 12 days. The MTHFR genotype of each colon carcinoma cell line is indicated in parentheses. The number of cells for each line was counted using the SRB assay at 3 time points. Each point represents the mean of 3 replicates ±SD.

FIG. 13 depicts cell survival and MTHFR protein levels after treatment with the antisense oligonucleotide EX5. (A) Cells were treated on three successive days with increasing concentration of EX5 (o). Cells were also treated with a control oligonucleotide, CTSEX5. The number of surviving cells was determined by SRB staining. Cell survival after transfection with EX5 is expressed as a % of survival after transfection with the control CTSEX5 oligonucleotide. Error bars represent ±SE of the mean of 3 experiments, each performed in triplicate. (B) MTHFR protein levels after three rounds of treatments with Lipofectin only (mock transfection), 400 nM of CTSEX5, 200 nM of EX5 or 400 nM of EX5. Cells were harvested after the third treatment and subjected to Western blot analysis. The position of the MTHFR protein and the molecular weight markers are indicated. Protein levels of β-actin were also assayed by Western blotting to verify equal loading of samples.

FIG. 14 depicts a comparison of the cell survival of normal human fibroblasts, breast carcinoma cells and neuroblastoma lines after treatment with 400 nM of EX5. Cells were treated three successive days with 400 nM of EX5 and 400 nM of CTSEX5. The number of surviving cells was determined by SRB staining as described in the Examples. For each cell line, cell survival after transfection with EX5 is expressed as a % of survival after transfection with the control CTSEX5 oligonucleotide. Each value on the graph represents the mean of three replicates ±SD.

FIG. 15 depicts the effect of treatment with the methylated antisense oligonucleotide SEQ ID NO:19 on SW620 colon tumor volume in CD-1 mice.

FIG. 16 depicts the effect of treatment with the methylated antisense oligonucleotide SEQ ID NO:19 on A549 lung tumor volume in CD-1 mice.

FIG. 17 depicts Western blotting analysis of MTHFR protein levels in A549 lung tumors after treatment with the methylated antisense oligonucleotide SEQ ID NO:19.

FIG. 18 depicts Western blotting analysis of PARP protein levels in A549 lung tumors after treatment with the methylated antisense oligonucleotide SEQ ID NO:19.

FIG. 19 depicts the effect of the methylated antisense oligonucleotide SEQ ID NO:19 on the survival of PC3 prostate carcinoma cells in vitro.

FIG. 20 depicts the effect of combinations of cisplatin (CDDP) and the methylated antisense oligonucleotide SEQ ID NO:19 on the survival of PC3 prostate carcinoma cells in vitro.

FIG. 21 depicts the effect of combinations of cisplatin (CDDP) and the methylated antisense oligonucleotide SEQ ID NO:19 on the survival of SK-N-F1 neuroblastoma cells in vitro.

FIG. 22 depicts the effect of combinations of cisplatin (CDDP) and the methylated antisense oligonucleotide SEQ ID NO:19 on the survival of MCF-7 breast cancer cells in vitro.

FIG. 23 depicts the effect of combinations of 5-fluorouracil (5-FU) and the antisense oligonucleotide SEQ ID NO:19 on the survival of MCF-7 breast carcinoma cells in vitro.

FIG. 24 depicts the effect of combinations of Taxol® and the methylated antisense oligonucleotide SEQ ID NO:19 on the survival of MCF-7 breast carcinoma cells in vitro.

FIG. 25 depicts the effect of combinations of cisplatin (CDDP) and the methylated antisense oligonucleotide SEQ ID NO:19 on the survival of A549 lung carcinoma cells in vitro.

FIG. 26 depicts the effect of combinations of 5-fluorouracil (5-FU) and the methylated antisense oligonucleotide SEQ ID NO:19 on the survival of SW620 colon carcinoma cells in vitro.

FIG. 27 depicts the effect of treatment with the methylated antisense oligonucleotide SEQ ID NO:19 in combination with cisplatin (CDDP) on A549 lung tumor volume in CD-1 mice.

FIG. 28 depicts the effect of treatment with the methylated antisense oligonucleotide SEQ ID NO:19 in combination with 5-fluorouracil (5-FU) on SW620 colon tumor volume in CD-1 mice.

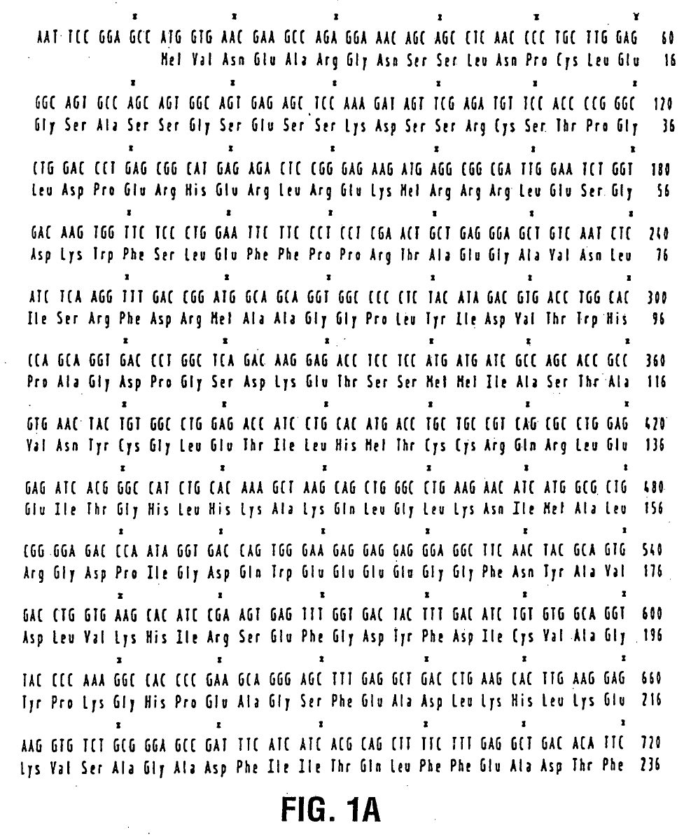

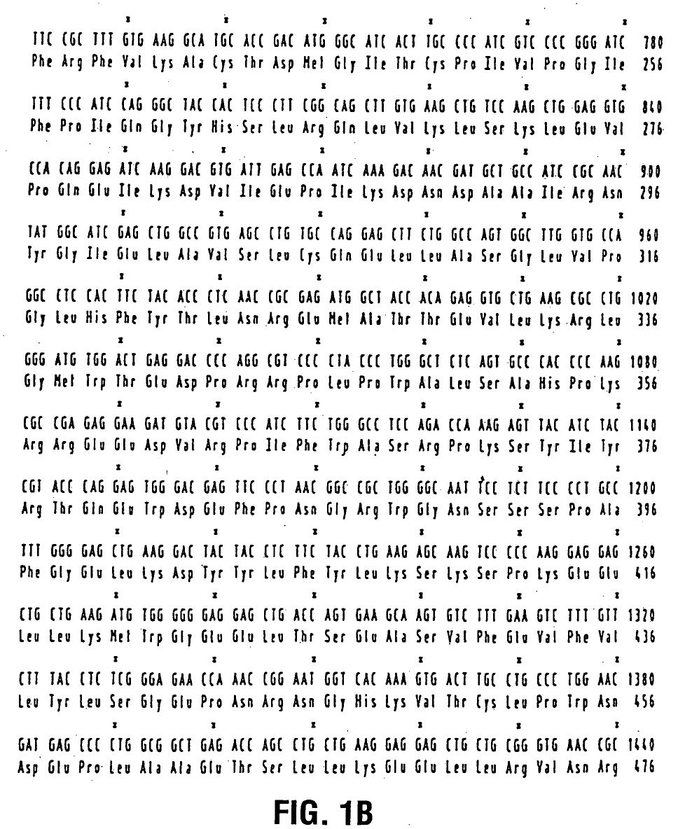

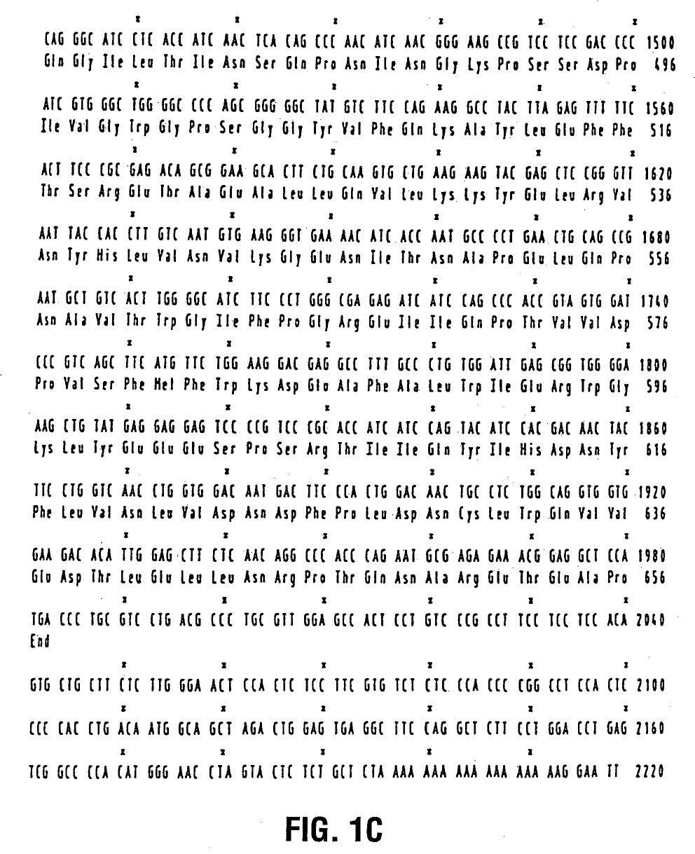

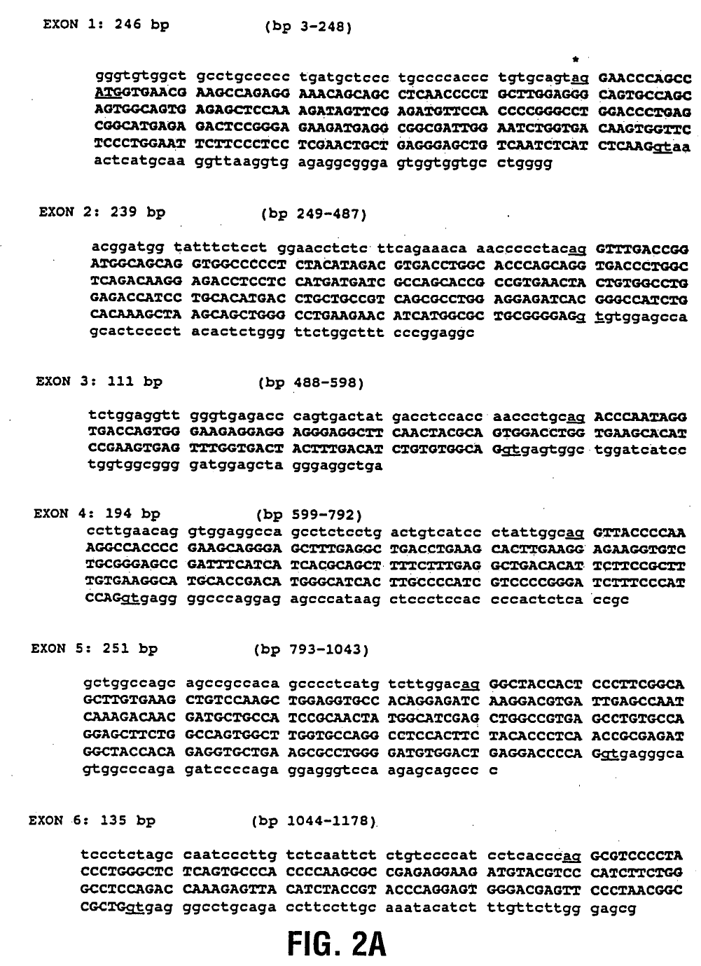

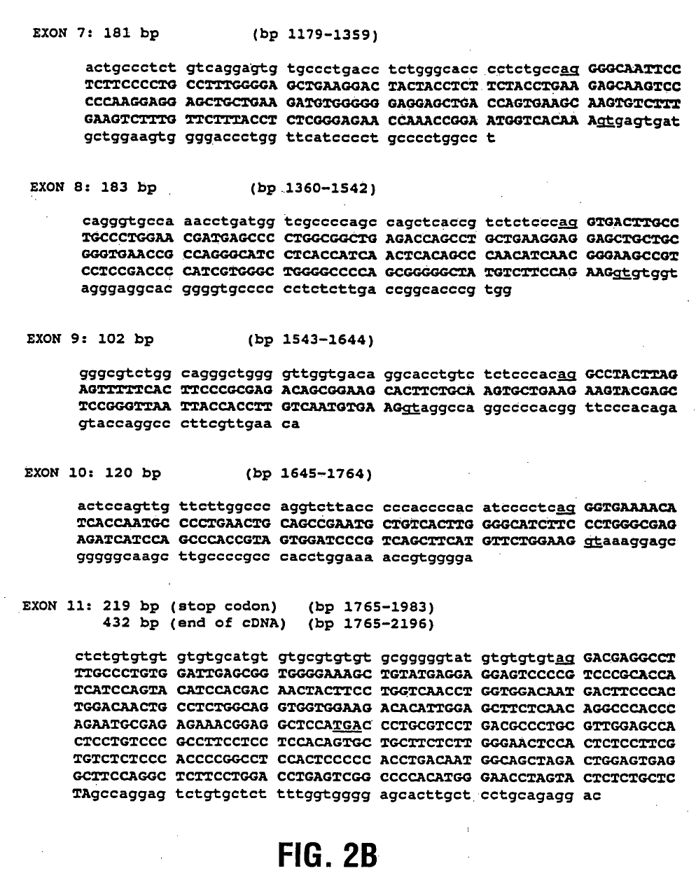

### DETAILED DESCRIPTION OF THE INVENTION

The present invention provides a method of selectively inhibiting the growth of cancer cells by downregulating MTHFR activity. In the context of the present invention, downregulation of MTHFR activity can be achieved by inhibition of MTHFR gene expression or by direct inhibition of the MTHFR protein.

In view of the prior art regarding the deleterious effects of MTHFR deficiency, a downregulation of levels of MTHFR activity would be expected to produce only negative effects. Therefore, a particularly unexpected result of downregulation of MTHFR protein levels in a mouse cancer model was its effectiveness in reducing the size of tumors. As described herein, this unexpected finding was further demonstrated following inhibition of MTHFR gene expression using non-allele specific antisense oligonucleotides in the treatment of cancer cells and of normal cells.

The present invention thus provides for methods of treating a variety of mammalian cancers by administration of an effective amount of one or more MTHFR inhibitors. The MTHFR inhibitors can be administered alone, or may be used in combination therapy, i.e. in conjunction with one or more standard chemotherapeutic agents in the treatment of cancer.

Definitions

Unless defined otherwise, all technical and scientific terms used herein have the same meaning as commonly understood by one of ordinary skill in the art to which this invention pertains.

The term “non allele-specific” as used herein refers to a compound capable of binding to at least two different MTHFR alleles.

The term “specifically hybridize” as used herein refers to the ability of a nucleic acid to bind detectably and specifically to a second nucleic acid. Polynucleotides, oligonucleotides and fragments thereof specifically hybridize to target nucleic acid strands under hybridization and wash conditions that minimize appreciable amounts of detectable binding to nonspecific nucleic acids. High stringency conditions can be used to achieve specific hybridization as known in the art (for example, see Ausubel, et al., (2000) Current Protocols in Molecular Biology, Wiley & Sons, New York, N.Y.).

Typically, hybridization and washing conditions are performed at high stringency according to conventional hybridization procedures. Washing conditions are typically 1-3×SSC, 0.1-1% SDS, 50-70° C., with a change of wash solution after about 5-30 minutes.

The term “corresponds to” as used herein with reference to nucleic acid sequences means a polynucleotide sequence that is identical to all or a portion of a reference polynucleotide sequence. In contradistinction, the term “complementary to” is used herein to mean that the polynucleotide sequence is identical to all or a portion of the complement of a reference polynucleotide sequence. For illustration, the nucleotide sequence “TATAC” corresponds to a reference sequence “TATAC” and is complementary to a reference sequence “GTATA.”

The following terms are used herein to describe the sequence relationships between two or more polynucleotides: “reference sequence,” “comparison window,” “sequence identity,” “percentage of sequence identity,” and “substantial identity.” A “reference sequence” is a defined sequence used as a basis for a sequence comparison; a reference sequence may be a subset of a larger sequence, for example, as a segment of a fill-length cDNA or gene sequence, or may comprise a complete cDNA or gene sequence. Generally, a reference sequence is at least 20 nucleotides in length, frequently at least 25 nucleotides in length, and often at least 50 nucleotides in length. Since two polynucleotides may each (1) comprise a sequence (i.e. a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity.

A “comparison window”, as used herein, refers to a conceptual segment of at least 20 contiguous nucleotide positions wherein a polynucleotide sequence may be compared to a reference sequence of at least 20 contiguous nucleotides and wherein the portion of the polynucleotide sequence in the comparison window may comprise additions or deletions (i.e. gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman (1981) Adv. Appl. Math. 2:482, by the homology alignment algorithm of Needleman and Wunsch (1970) J. Mol. Biol. 48:443, by the search for similarity method of Pearson and Lipman (1988) Proc. Natl. Acad. Sci. (U.S.A.) 85:2444, by computerized implementations of these algorithms (GAP, BESTFIT, FASTA, and TFASTA in the Wisconsin Genetics Software Package Release 7.0, Genetics Computer Group, 573 Science Dr., Madison, Wis.), or by inspection, and the best alignment (i.e. resulting in the highest percentage of homology over the comparison window) generated by the various methods is selected.

The term “sequence identity” means that two polynucleotide sequences are identical (i.e. on a nucleotide-by-nucleotide basis) over the window of comparison. The term “percentage of sequence identity” is calculated by comparing two optimally aligned sequences over the window of comparison, determining the number of positions at which the identical nucleic acid base (e.g. A, T, C, G, U, or I) occurs in both sequences to yield the number of matched positions, dividing the number of matched positions by the total number of positions in the window of comparison (i.e. the window size), and multiplying the result by 100 to yield the percentage of sequence identity.

The term “substantial identity” as used herein denotes a characteristic of a polynucleotide sequence, wherein the polynucleotide comprises a sequence that has at least 30 percent sequence identity, often at least 50 percent sequence identity, and more usually at least 60 percent sequence identity as compared to a reference sequence over a comparison window of at least 20 nucleotide positions, frequently over a window of at least 25-50 nucleotides, wherein the percentage of sequence identity is calculated by comparing the reference sequence to the polynucleotide sequence which may include deletions or additions which total 20 percent or less of the reference sequence over the window of comparison.

Administration of an MTHFR inhibitor “in combination with” one or more chemotherapeutic agents, is intended to include simultaneous (concurrent) administration and consecutive administration. Consecutive administration is intended to encompass administration of the chemotherapeutic agent(s) and the MTHFR inhibitor(s) to the subject in various orders.

The term “subject” or “patient” as used herein refers to a mammal in need of treatment.

As used herein, the term “about” refers to a +/−10% variation from the nominal value. It is to be understood that such a variation is always included in any given value provided herein, whether or not it is specifically referred to.

MTHFR Inhibitors

The present invention provides compounds that selectively inhibit the growth of cancer cells by downregulating MTHFR activity in a mammal (e.g., a human). The extent of selective inhibition is generally sufficient to treat, stabilize, or prevent cancer in the mammal. In the context of the present invention, selective inhibition means that the growth of cancer cells is inhibited substantially more than the growth of normal cells. In a specific embodiment of the present invention cancer cell growth is inhibited by an MTHFR inhibitor under conditions in which the growth of normal cells also treated with the MTHFR inhibitor is fully or partially unaffected. When the growth of normal cells is partially affected by contact with the MTHFR inhibitor, the difference between the effect on cancer cells and on normal cells is such that the cancer cells are preferentially inhibited and/or killed by contact with the MTHFR inhibitor.

The inhibitors according to the present invention can be oligonucleotide inhibitors such as antisense oligonucleotides (including triple helix oligonucleotides and siRNA molecules) and ribozymes, or they can be biologically inactive MTHFR proteins or fragments, peptides, small molecule inhibitors or antibodies.

(i) Antisense Oligonucleotides

In one embodiment of the invention, the MTHFR inhibitors are antisense oligonucleotides targeted to a mammalian MTHFR gene. “Targeting” an antisense compound to a particular nucleic acid, in the context of this invention, is a multistep process. The process usually begins with the identification of a nucleic acid sequence whose function is to be modulated. In the present invention, the target is the gene encoding MTHFR. The targeting process also includes determination of a site or sites within this gene for the antisense interaction to occur such that the desired effect, i.e. modulation of expression of the protein encoded by the gene, will result.

Generally, there are five regions of a gene that may be targeted for antisense modulation: the 5′ untranslated region (5′-UTR), the translation initiation or start codon region, the open reading frame (ORF), the translation termination or stop codon region and the 3′ untranslated region (3′-UTR).

The terms “translation initiation codon” and “start codon” can encompass many codon sequences, even though the initiator amino acid in each instance is typically methionine in eukaryotes. It is also known in the art that eukaryotic genes may have two or more alternative start codons, any one of which may be preferentially utilized for translation initiation in a particular cell type or tissue, or under a particular set of conditions. In the context of the present invention, “start codon” and “translation initiation codon” refer to the codon or codons that are used in vivo to initiate translation of an mRNA molecule transcribed from a gene encoding MTHFR regardless of the sequence(s) of such codons.

As is known in the art, some eukaryotic transcripts are directly translated, however, most mammalian ORFs contain one or more sequences, known as “introns,” which are excised from a transcript before it is translated; the expressed (unexcised) portions of the ORF are referred to as “exons” (Alberts et al., (1983) Molecular Biology of the Cell, Garland Publishing Inc., New York, pp. 411-415). In the context of the present invention, both introns and exons may serve as targets for antisense.

In some instances, an ORF may also contain one or more sites that may be targeted for antisense due to some functional significance in vivo. Examples of the latter types of sites include intragenic stem-loop structures (see, for example, U.S. Pat. No. 5,512,438) and, in unprocessed mRNA molecules, intron/exon splice sites. In addition, mRNA molecules possess a 5′ cap region that may also serve as a target for antisense. The 5′ cap of a mRNA comprises an N⁷-methylated guanosine residue joined to the 5′-most residue of the mRNA via a 5′-5′ triphosphate linkage. The 5′ cap region of a mRNA is considered to include the 5′ cap structure itself as well as the first 50 nucleotides adjacent to the cap.

In accordance with the present invention, the antisense oligonucleotides are non allele-specific, therefore, regions of the gene to be targeted are those that are conserved, i.e. show no sequence difference, among the different alleles of the MTHFR gene. In one embodiment of the present invention, the antisense oligonucleotides are targeted to all or part of one of exons 1-11 or other MTHFR exon (Goyette et al., (1998) Mammalian Genome 9:652-656). In another embodiment, the antisense oligonucleotides are targeted to exon 5 of the MTHFR gene. In a further embodiment of the present invention, the antisense oligonucleotides comprise the sequence 5′-AGCTGCCGAAGGGAGTGGTA-3′ (SEQ ID NO:16) and bind to nucleotides 796-815 of exon 5 of the MTHFR gene or the mRNA transcribed therefrom.

In accordance with the present invention, the antisense oligonucleotide binds at least 70% of the human MTHFR alleles. In a related embodiments the antisense oligonucleotide binds at least 80%, or at least 90% of the human MTHFR alleles. In another embodiment of the present invention, the antisense nucleic acid does not bind to a region of the MTHFR gene that contains a polymorphic site.

Once the target site or sites have been identified, oligonucleotides are chosen that are sufficiently complementary (i.e. hybridize with sufficient strength and specificity) to the target to give the desired result.

The antisense oligonucleotides in accordance with the present invention are selected from a sequence complementary to the MTHFR gene such that the sequence exhibits the least likelihood of forming duplexes, hair-pins, or of containing homooligomer/sequence repeats. The oligonucleotide may further contain a GC clamp. These properties can be determined qualitatively using commercially available computer software, for example, the computer modeling program OLIGO® Primer Analysis Software, Version 5.0 (distributed by National Biosciences, Inc., Plymouth, Minn.).

In order to be effective, antisense oligonucleotides are typically between 7 and 350 nucleotides in length. In one embodiment of the present invention the antisense oligonucleotides comprise from about 7 to about 100 nucleotides, or nucleotide analogues. In another embodiment, the antisense oligonucleotides comprise from about 7 to about 50 nucleotides, or nucleotide analogues. In other embodiments the antisense oligonucleotides comprise from about 10 to about 35, from about 15 to about 25 and from about 18 to about 22 nucleotides, or nucleotide analogues.

In a specific embodiment, the antisense oligonucleotides comprise at least 7 consecutive nucleotides of a sequence complementary to a portion of exon 5 of an MTHFR gene or mRNA. In another embodiment, the antisense oligonucleotides comprise at least 7 consecutive nucleotides of the sequence set forth in SEQ ID NO:16 or 19.

It is understood in the art that an antisense oligonucleotide need not have 100% identity with its target sequence. The present invention, therefore, contemplates antisense oligonucleotides that have 100% sequence identity with the target sequence as well as those that have a sequence that is at least about 75% identical to the target sequence. In one embodiment of the present invention, the antisense oligonucleotides have a sequence that is at least about 90% identical. In a related embodiment, they have a sequence that is at least about 95% identical with the target sequence, allowing for gaps or mismatches of several bases. In accordance with the present invention, the antisense oligonucleotide is less than 50% identical to the reverse complement of a region in another human expressed sequence (EST) or, in other reported human ESTs. Identity can be determined, for example, by using the BLASTN program of the University of Wisconsin Computer Group (GCG) software.

Alternatively an antisense oligonucleotide of the present invention can be defined by its ability to specifically hybridize to the target MTHFR gene, as determined using standard techniques known to workers skilled in the art (e.g. hybridization assays).

In the context of the present invention, the term “oligonucleotide” refers to an antisense oligomer or polymer of ribonucleic acid (RNA), deoxyribonucleic acid (DNA), modified RNA or DNA, or RNA or DNA mimetics. This term, therefore, includes oligonucleotides composed of naturally-occurring nucleobases, sugars and covalent internucleoside (backbone) linkages as well as oligonucleotides having non-naturally-occurring portions which function similarly. Such modified or substituted oligonucleotides are often preferred over native forms because of desirable properties such as, enhanced cellular uptake, enhanced affinity for nucleic acid target and increased stability in the presence of nucleases.

Examples of modified or substituted antisense compounds useful in this invention include oligonucleotides containing modified backbones or non-natural internucleoside linkages. In accordance with the present invention, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Exemplary modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Exemplary modified oligonucleotide backbones that do not include a phosphorus atom are formed by short chain alkyl or cycloalkyl internucleoside linkages, mixed heteroatom and alkyl or cycloalkyl internucleoside linkages, or one or more short chain heteroatomic or heterocyclic internucleoside linkages. Such backbones include morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

The present invention also contemplates oligonucleotide mimetics in which both the sugar and the internucleoside linkage of the nucleotide units are replaced with novel groups. The base units are maintained for hybridization with an appropriate nucleic acid target compound. An example of such an oligonucleotide mimetic, which has been shown to have excellent hybridization properties, is a peptide nucleic acid (PNA) (Nielsen et al., (1991) Science, 254:1497-1500). In PNA compounds, the sugar-backbone of an oligonucleotide is replaced with an amide containing backbone, in particular an aminoethylglycine backbone. The nucleobases are retained and are bound directly or indirectly to aza-nitrogen atoms of the amide portion of the backbone.

Modified oligonucleotides may also contain one or more substituted sugar moieties. For example, oligonucleotides may comprise sugars with one of the following substituents at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Examples of such groups are: O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Alternatively, the oligonucleotides may comprise one of the following substituents at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Specific examples include 2′-methoxyethoxy (2′-O═CH₂CH₂OCH₃, also known as 2′-O-(2-methoxyethyl) or 2′-MOE) (Martin et al., (1995) Helv. Chim. Acta, 78:486-504), 2′-dimethylaminooxyethoxy (O(CH₂)₂ON(CH₃)₂ group, also known as 2′-DMAOE), 2′-methoxy (2′-O═CH₃), 2′-aminopropoxy (2′-OCH₂CH₂CH₂NH₂) and 2′-fluoro (2′-F).

Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar.

Oligonucleotides may also include modifications or substitutions to the nucleobase. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808; The Concise Encyclopedia Of Polymer Science And Engineering, (1990) pp 858-859, Kroschwitz, J. I., ed. John Wiley & Sons; Englisch et al., (1991) Angewandte Chemie, Int. Ed., 30:613; and Sanghvi, Y. S., (1993) Antisense Research and Applications, pp 289-302, Crooke, S. T. and Lebleu, B., ed., CRC Press. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., (1993) Antisense Research and Applications, pp 276-278, Crooke, S. T. and Lebleu, B., ed., CRC Press, Boca Raton).

Oligonucleotides may also comprise one or more methylated cytosine residues. As is known in the art, methylation of the cytosine in a CpG motif can affect the immunostimulatory activity of antisense oligonucleotides (see, for example, Krieg et al., (1995) Nature 374:546-549).

Another oligonucleotide modification included in the present invention is to chemically link to the oligonucleotide one or more moieties or conjugates which enhance the activity, cellular distribution or cellular uptake of the oligonucleotide. Such moieties include, but are not limited to, lipid moieties such as a cholesterol moiety (Letsinger et al., (1989) Proc. Natl. Acad. Sci. USA, 86:6553-6556), cholic acid (Manoharan et al., (1994) Bioorg. Med. Chem. Let., 4:1053-1060), a thioether, e.g. hexyl-S-tritylthiol (Manoharan et al., (1992) Ann. N. Y. Acad. Sci., 660:306-309; Manoharan et al., (1993) Bioorg. Med. Chem. Lett., 3:2765-2770), a thiocholesterol (Oberhauser et al., (1992) Nucl. Acids Res., 20:533-538), an aliphatic chain, e.g. dodecandiol or undecyl residues (Saison-Behmoaras et al., (1991) EMBO J., 10:1111-1118; Kabanov et al., (1990) FEBS Lett., 259:327-330; Svinarchuk et al., (1993) Biochimie, 75:49-54), a phospholipid, e.g. di-hexadecyl-rac-glycerol or triethylammonium 1,2-di-O-hexadecyl-rac-glycero-3-H-phosphonate (Manoharan et al., (1995) Tetrahedron Lett., 36:3651-3654; Shea et al., (1990) Nucl. Acids Res., 18:3777-3783), a polyamine or a polyethylene glycol chain (Manoharan et al., (1995) Nucleosides & Nucleotides, 14:969-973), or adamantane acetic acid (Manoharan et al., (1995) Tetrahedron Lett., 36:3651-3654), a palmityl moiety (Mishra et al., (1995) Biochim. Biophys. Acta, 1264:229-237), or an octadecylamine or hexylamino-carbonyl-oxycholesterol moiety (Crooke et al., (1996) J. Pharmacol. Exp. Ther., 277:923-937).

One skilled in the art will recognise that it is not necessary for all positions in a given oligonucleotide to be uniformly modified. The present invention, therefore, contemplates the incorporation of more than one of the aforementioned modifications into a single oligonucleotide or even at a single nucleoside within the oligonucleotide. The present invention further includes antisense compounds that are chimeric compounds. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

In the context of the present invention, an antisense oligonucleotide is “nuclease resistant” when it has either been modified such that it is not susceptible to degradation by DNA and RNA nucleases or alternatively has been placed in a delivery vehicle which in itself protects the oligonucleotide from DNA or RNA nucleases. Nuclease resistant oligonucleotides include, for example, methyl phosphonates, phosphorothioates, phosphorodithioates, phosphotriesters, and morpholino oligomers. Suitable delivery vehicles for conferring nuclease resistance include, for example, liposomes.

The present invention further contemplates antisense oligonucleotides that contain groups for improving the pharmacokinetic properties of the oligonucleotide, or groups for improving the pharmacodynamic properties of the oligonucleotide.

In one embodiment of the present invention, the antisense oligonucleotide is a phosphorothioate oligonucleotide in which a non-bridging phosphoryl oxygen in one or more of the nucleotides is replaced with sulphur. In another embodiment, all the backbone linkages in the antisense oligonucleotide are phosphorothioate linkages. In a further embodiment, the antisense oligonucleotide is a phosphorothioate oligonucleotide comprising at least 7 consecutive nucleotides of the sequence 5′-AGCTGCCGAAGGGAGTGGTA-3′ (SEQ ID NO:16).

In another embodiment of the invention, the antisense oligonucleotide comprises at least CpG motif in which the cytosine is methylated. In a further embodiment, the antisense oligonucleotide comprises at least 7 consecutive nucleotides of the sequence 5′-AGCTGCcGAAGGGAGTGGTA-3′ (SEQ ID NO:19), wherein the lowercase “c” represents a methylated cytosine. Combinations of one or more phosphorothioate backbone linkages and one or more methylated cytosine residues in a single oligonucleotide are also contemplated.

The antisense oligonucleotides of the present invention can be prepared by conventional techniques well-known to those skilled in the art. For example, the oligonucleotides can be prepared using solid-phase synthesis using commercially available equipment, such as the equipment available from Applied Biosystems Canada Inc., Mississauga, Canada. As is well-known in the art, modified oligonucleotides, such as phosphorothioates and alkylated derivatives, can also be readily prepared by similar methods.

Alternatively, the antisense oligonucleotides of the present invention can be prepared by enzymatic digestion of the naturally occurring MTHFR gene by methods known in the art.

Antisense oligonucleotides can also be prepared by recombinant DNA techniques. The present invention, therefore, encompasses expression vectors comprising nucleic acid sequences that encode one or more antisense oligonucleotide that targets the MTHFR gene. The antisense oligonucleotide(s) encoded by such expression vectors is expressed in a suitable host cell. Suitable expression vectors can be readily constructed using procedures known in the art. Examples of suitable vectors include, but are not limited to, plasmids, phagemids, cosmids, bacteriophages, baculoviruses, retroviruses, and RNA and DNA viruses. Generally, the viral vectors are replication deficient by are capable of expression f the antisense oligonucleotide(s).

One skilled in the art will understand that selection of the appropriate host cell for expression of the antisense oligonucleotide will be dependent upon the vector chosen. Examples of host cells include, but are not limited to, bacterial, yeast, insect, plant and mammalian cells.

One skilled in the art will also understand that the expression vector may further include regulatory elements required for efficient transcription or translation of the antisense oligonucleotide sequences. Examples of regulatory elements that can be incorporated into the vector include, but are not limited to, transcriptional elements such as promoters, enhancers, terminators, and polyadenylation signals. The present invention, therefore, provides vectors comprising one or more regulatory elements operatively linked to a nucleic acid sequence encoding an antisense oligonucleotide. One skilled in the art will appreciate that selection of suitable regulatory elements is dependent on the host cell chosen for expression of the antisense oligonucleotide and that such elements may be derived from a variety of sources, including bacterial, fungal, viral, mammalian or insect genes.

In the context of the present invention, the expression vector may additionally contain a reporter gene. Suitable reporter genes include, but are not limited to, β-galactosidase, green fluorescent protein, red fluorescent protein, luciferase, and β-glucuronidase. Incorporation of a reporter gene into the expression vector allows transcription of the antisense oligonucleotide to be monitored by detection of a signal generated by expression of the reporter gene.

In accordance with the present invention, the expression vectors can be introduced into a suitable host cell or tissue by one of a variety of methods known in the art. These methods include, for example, stable or transient transfection, lipofection, electroporation, and infection with recombinant viral vectors. Methods of constructing expression vectors and introducing these vectors into host cells are well-known in the art, and are generally described in Sambrook et al., (1992) Molecular Cloning: A Laboratory Manual, Cold Spring Harbor Press; Ausubel et al., (2000) Current Protocols in Molecular Biology, Wiley & Sons, New York.

The MTHFR inhibitors can also be oligonucleotides that hybridizes to and form triple helix structures at the 5′ terminus of the MTHFR gene thus blocking transcription. Such “triple helix forming” oligonucleotides can be prepared as described above in relation to the antisense oligonucleotides. Similarly, nucleic acids encoding the triple helix forming oligonucleotide can be cloned into a vector as described above.

The antisense oligonucleotides of the invention can also be provided as short interfering RNA (siRNA) molecules. RNA interference mediated by double-stranded siRNA molecules is known in the art to play an important role in post-transcriptional gene silencing [Zamore, Nature Struc. Biol., 8:746-750 (2001)]. siRNA molecules are typically 21-22 base pairs in length and are generated in nature when long double-stranded RNA molecules are cleaved by the action of an endogenous ribonuclease. Recently, it has been demonstrated that transfection of mammalian cells with synthetic siRNA molecules having a sequence identical to a target gene leads to a reduction in the mRNA levels of the target gene [Elbashir, et al., Nature, 411:494-498 (2001)]. Single-stranded siRNA molecules have also been described.

When the antisense oligonucleotides of the invention are provided as synthetic siRNA molecules, they can be double-stranded or single stranded RNA molecules. As is known in the art, effective siRNA molecules need to be less than 30 base pairs in length to prevent them triggering non-specific RNA interference pathways in the cell via the interferon response. Thus, in one embodiment of the present invention, the siRNA molecules are between about 7 and about 30 base pairs in length. In other embodiments, they are between about 10 and about 25 and between about 15 and about 25 base pairs in length. It is also known in the art that effective siRNA molecules can be developed based on sequences that have been shown to have activity as standard antisense oligonucleotides. Thus, in one embodiment of the invention, the siRNA molecules target exon 5 of an MTHFR gene. In another embodiment, the siRNA molecules comprise at least 7 consecutive nucleotides of the sequence as set forth in SEQ ID NO:16.

Double-stranded siRNA molecules can further comprise poly-T or poly-U overhangs at each end to minimise RNase-mediated degradation of the molecules, for example, overhangs at the 3′ and 5′ ends which consist of two thymidine or two uridine residues. Design and construction of siRNA molecules is known in the art [see, for example, Elbashir, et al., Nature, 411:494-498 (2001); Bitko and Barik, BMC Microbiol., 1:34 (2001)]. In addition, kits that provide a rapid and efficient means of constructing siRNA molecules by in vitro transcription are also commercially available (for example, from Ambion, Austin, Tex.; New England Biolabs, Beverly, Mass.).

The ability of the antisense oligonucleotides, triple helix forming oligonucleotides and siRNA molecules of the present invention to inhibit MTHFR gene expression can be determined by a number of techniques known to one skilled in the art. For example, cells that normally express MTHFR can be treated with an oligonucleotide inhibitor and the level of MTHFR mRNA can subsequently be determined by standard Northern blot analysis, and/or the level of MTHFR protein can be determined by standard Western blot analysis. Methods of conducting these techniques are well-known to workers skilled in art (see, for example, Ausubel et al., (2000) Current Protocols in Molecular Biology, Wiley & Sons, New York: Coligan, et al., (2001) Current Protocols in Protein Science, Wiley & Sons, New York). In one embodiment of the present invention, the level of MTHFR protein is determined by measuring the level of MTHFR enzymatic activity, as described in Christensen, et al., (1997) Arterioscler. Thromb. Vasc. Bio., 17:573-596. In an alternate embodiment, the level of MTHFR protein can be assessed by measuring the resulting increase in cellular levels of homocysteine, or decrease in 5-methyltetrahydrofolate or methionine, as described herein.

(ii) Ribozymes

In one embodiment of the present invention the MTHFR inhibitor is a ribozyme that specifically targets RNA encoding MTHFR. Ribozymes are RNA molecules having an enzymatic activity which is able to repeatedly cleave other separate RNA molecules in a nucleotide base sequence specific manner. Such enzymatic RNA molecules can be targeted to virtually any RNA transcript, and efficient cleavage achieved in vitro (see, for example Kim et al., (1987) Proc. Natl. Acad. Sci. USA 84:8788; Haseloff and Gerlach, (1988) Nature 334:585; Cech (1988) JAMA 260:3030; and Jefferies et al., (1989) Nucleic Acids Research 17:1371).

Ribozymes act by first binding to a target RNA. Such binding occurs through the target RNA binding portion of a ribozyme which is held in close proximity to an enzymatic portion of the RNA which acts to cleave the target RNA. Thus, the ribozyme first recognizes and then binds a target RNA through complementary base-pairing, and once bound to the correct site, acts enzymatically to cut the target RNA. Strategic cleavage of such a target RNA will destroy its ability to direct synthesis of an encoded protein. After a ribozyme has bound and cleaved its RNA target it is released from that RNA to search for another target and can repeatedly bind and cleave new targets.

Hammerhead ribozymes comprise a hybridizing region which is complementary in nucleotide sequence to at least part of the target RNA, and a catalytic region which is adapted to cleave the target RNA. The hybridizing region contains nine (9) or more nucleotides. Therefore, the hammerhead ribozymes of the present invention have a hybridizing region which is complementary to a gene encoding MTHFR and is at least nine nucleotides in length. The construction and production of such ribozymes is well known in the art and is described more fully in Haseloff and Gerlach, (1988) Nature 334:585-591.

The ribozymes of the present invention also include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (Zaug, et al., (1984) Science 224:574-578; Zaug and Cech, (1986) Science 231:470-475; Zaug, et al., 1986, Nature, 324:429-433; published International Patent Application No. WO 88/04300 by University Patents Inc.; Been and Cech, (1986) Cell 47:207-216). The Cech endoribonucleases have an eight base pair active site which hybridizes to a target RNA sequence whereafter cleavage of the target RNA takes place.

There is a narrow range of binding free-energies between a ribozyme and its substrate that will produce maximal ribozyme activity. Such binding energy can be optimized by making ribozymes with G to I and U to BrU substitutions (or equivalent substitutions) in the substrate-binding arms. This allows manipulation of the binding free-energy without actually changing the target recognition sequence, the length of the two substrate-binding arms, or the enzymatic portion of the ribozyme. The shape of the free-energy vs. ribozyme activity curve can be readily determined using data from experiments in which each base (or several bases) is modified or unmodified, and without the complication of changing the size of the ribozyme/substrate interaction.

Such experiments will indicate the most active ribozyme structure. The use of modified bases thus permits “fine tuning” of the binding free energy to assure maximal ribozyme activity. In addition, replacement of such bases, e.g., I for G, may permit a higher level of substrate specificity when cleavage of non-target RNA is a problem.

The ability of the ribozymes of the present invention to inhibit MTHFR mRNA expression can be determined by a number of techniques known to one skilled in the art. For example, the level of MTHFR protein can be determined by standard Western blot analysis. Techniques of conducting this method are well-known to workers skilled in art (see, for example, Ausubel et al., (2000) Current Protocols in Molecular Biology, Wiley & Sons, New York: Coligan, et al., (2001) Current Protocols in Protein Science, Wiley & Sons, New York). In one embodiment of the present invention, the level of MTHFR protein is determined by measuring the level of MTHFR enzymatic activity, as described in Christensen, et al., (1997) Arterioscler. Thromb. Vasc. Bio., 17:573-596. In an alternate embodiment, the level of MTHFR protein can be assessed by measuring the resulting increase in cellular levels of homocysteine, or decrease in 5-methyltetrahydrofolate or methionine, as described herein.

(iii) Biologically Inactive MTHFR Protein or Fragments of an MTHFR Protein

The present invention also contemplates the use of a biologically inactive MTHFR proteins or fragments of an MTHFR protein that interfere with the action of the wild-type protein and thus, acts as inhibitors of MTHFR activity.

Candidate inhibitory fragments can be selected from random fragments generated from the wild-type MTHFR protein. Methods for generating the candidate polypeptide fragments are well known to workers skilled in the art and include, but are not limited to, enzymatic, chemical or mechanical cleavage of the native protein, expression of nucleic acids encoding such fragments, etc. Biologically inactive MTHFR proteins can be generated by a variety of techniques known to a worker skilled in the art. For example, by site-directed or random mutagenesis techniques of nucleic acids encoding the protein, or by inactivation of the protein by chemical or physical means.

The ability of the biologically inactive MTHFR proteins or fragments to interfere with the wild-type MTHFR activity can be determined by standard techniques, for example, using the method described by Christensen, et al., (1997) Arterioscler. Thromb. Vasc. Bio., 17:573-596, or competitive binding studies.

(iv) Peptide Inhibitors

The present invention also provides for polypeptides and peptides that bind to and inhibit the activity of the MTHFR protein. One exemplary method of identifying such peptides is by phage display techniques. Phage display libraries of random short peptides are commercially available, e.g. from New England Biolabs, Inc., and are utilized through an in vitro selection process known as “panning”. In its simplest form, panning involves first incubating the library of phage displayed peptides with a plate, or bead, coated with the target molecule, then washing away unbound phage particles, and finally eluting the specifically bound phage. For the purposes of the present invention, the target molecule is the MTHFR protein, or a fragment thereof.

The peptide(s) displayed by the specifically-binding phage are then isolated and sequenced by standard techniques known to those skilled in the art. In some instances the binding strength of the isolated peptide is then tested using standard techniques. The ability of the peptides to inhibit MTHFR activity can also be determined using assays known in the art and as described herein.

(v) Small Molecule Inhibitors

Potential inhibitory compounds are screened from large libraries of synthetic or natural compounds. Numerous means are currently used for random and directed synthesis of saccharide, peptide, and nucleic acid based compounds and are well-known in the art. Synthetic compound libraries are commercially available from a number of companies including Maybridge Chemical Co. (Trevillet, Cornwall, UK), Comgenex (Princeton, N.J.), Brandon Associates (Merrimack, N.H.), and Microsource (New Milford, Conn.). A rare chemical library is available from Aldrich (Milwaukee, Wis.). Combinatorial libraries are also available and can be prepared according to standard procedures. Alternatively, libraries of natural compounds in the form of bacterial, fungal, plant, and animal extracts are available from, e.g., Pan Laboratories (Bothell, Wash.) or MycoSearch (North Carolina), or are readily producible. Additionally, natural and synthetically produced libraries and compounds are readily modified through conventional chemical, physical, and biochemical means.

These libraries can be screened for their ability to inhibit the activity of the MTHFR protein (for example, using the method described by Christensen, et al., (1997) Arterioscler. Thromb. Vasc. Bio., 17:573-596) or to inhibit expression of the MTHFR gene by techniques known in the art, e.g. nucleic acid binding assays, gel shift assays, and the like.

(vi) Antibodies

The present invention also contemplates the use of antibodies, and antibody fragments, raised against the MTHFR protein, or fragments thereof, as inhibitors of MTHFR activity.

For the production of antibodies, various hosts including goats, rabbits, rats, mice, humans, and others can be immunized by injection with the MTHFR protein, or with a fragment or oligopeptide thereof that has immunogenic properties. Depending on the host species, various adjuvants may be used to increase immunological response. Such adjuvants include, but are not limited to, Freund's adjuvant, mineral gels such as aluminum hydroxide, and surface active substances such as lysolecithin, pluronic polyols, polyanions, peptides, oil emulsions, Keyhole limpet hemolysin (KLH), and dinitrophenol. Examples of adjuvants used in humans include, BCG (bacilli Calmette-Guerin) and Corynebacterium parvum.

The oligopeptides, peptides, or fragments used to induce antibodies to MTHFR can have an amino acid sequence consisting of as little as about 5 amino acids. In one embodiment of the present invention, amino acid sequences of at least about 10 amino acids are used. These oligopeptides, peptides, or fragments can be identical to a portion of the amino acid sequence of the natural protein that contains the entire amino acid sequence of a small, naturally occurring molecule. If required, short stretches of MTHFR amino acids can be fused with those of another protein, such as KLH, and antibodies to the chimeric molecule can be produced.

Monoclonal antibodies to MTHFR can be prepared using techniques that provide for the production of antibody molecules by continuous cell lines in culture. These include, but are not limited to, the hybridoma technique, the human B-cell hybridoma technique, and the EBV-hybridoma technique (see, for example, Kohler, G. et al. (1975) Nature 256:495-497; Kozbor, D. et al. (1985) J. Immunol. Methods 81:31-42; Cote, R. J. et al. (1983) Proc. Natl. Acad. Sci. USA, 80:2026-2030; and Cole, S. P. et al. (1984) Mol. Cell Biol. 62:109-120). For example, the monoclonal antibodies according to the present invention can be obtained by immunizing animals, such as mice or rats, with purified MTHFR. Spleen cells isolated from the immunized animals are then immortalized using standard techniques. Those isolated immortalized cells whose culture supernatant contains an antibody that causes an inhibition of the activity of MTHFR with an IC₅₀ of less than 100 ng/ml are then selected and cloned using techniques that are familiar and known to one skilled in the art. The monoclonal antibodies produced by these clones are then isolated according to standard protocols.

The immortalization of the spleen cells of the immunized animals can be carried out by fusing these cells with a myeloma cell line, such as P3X63-Ag 8.653 (ATCC CRL 1580) according to the method in (1980) J. Imm. Meth. 39:285-308. Other methods known to a person skilled in the art can also be used to immortalize spleen cells. In order to detect immortalized cells that produce the desired antibody against the MTHFR protein, a sample of the culture supernatant is tested using an enzyme linked immunosorbent assay (ELISA) for reactivity with MTHFR. In order to obtain those antibodies that inhibit the enzymatic activity of MTHFR, the culture supernatant of clones that produce antibodies that bind to MTHFR is additionally examined for inhibition of MTHFR activity using an appropriate assay, such as those described herein. Those clones whose culture supernatant shows the desired inhibition of MTHFR activity are expanded and the antibodies produced by these clones are isolated according to known methods.

In addition, techniques developed for the production of “chimeric antibodies,” such as the splicing of mouse antibody genes to human antibody genes to obtain a molecule with appropriate antigen specificity and biological activity, can be used (Morrison, S. L. et al. (1984) Proc. Natl. Acad. Sci. 81:6851-6855; Neuberger, M. S. et al. (1984) Nature 312:604-608; and Takeda, S. et al. (1985) Nature 314:452-454). Alternatively, techniques described for the production of single chain antibodies can be adapted, using methods known in the art, to produce MTHFR-specific single chain antibodies. Antibodies with related specificity, but of distinct idiotypic composition, can be generated by chain shuffling from random combinatorial immunoglobulin libraries (see, for example, Burton D. R. (1991) Proc. Natl. Acad. Sci. USA, 88:10134-10137).

Antibodies can also be produced by inducing in vivo production in the lymphocyte population or by screening immunoglobulin libraries or panels of highly specific binding reagents as disclosed in the literature (Orlandi, R. et al. (1989) Proc. Natl. Acad. Sci. 86: 3833-3837; Winter, G. et al. (1991) Nature 349:293-299).

Antibody fragments which contain specific binding sites for MTHFR can also be generated. For example, such fragments include, but are not limited to, F(ab′)2 fragments produced by pepsin digestion of the antibody molecule and Fab fragments generated by reducing the disulphide bridges of the F(ab′)2 fragments. Alternatively, Fab expression libraries can be constructed to allow rapid and easy identification of monoclonal Fab fragments with the desired specificity (see, for example, Huse, W. D. et al. (1989) Scienc,e 246:1275-1281).

Various immunoassays can be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between MTHFR and its specific antibody. Examples of such techniques include ELISAs, radioimmunoassays (RIAs), and fluorescence activated cell sorting (FACS). Alternatively, a two-site, monoclonal-based immunoassay utilizing monoclonal antibodies reactive to two non-interfering MTHFR epitopes, or a competitive binding assay can be used (see, Maddox, D. E. et al. (1983) J. Exp. Med. 158:1211-1216). These and other assays are well known in the art (see, for example, Hampton, R. et al. (1990) Serological Methods: A Laboratory Manual, APS Press, St Paul, Minn., Section IV; Coligan, J. E. et al. (1997, and periodic supplements) Current Protocols in Immunology, Wiley & Sons, New York, N.Y.; Maddox, D. E. et al. (1983) J. Exp. Med. 158:1211-1216).

Selection of MTHFR Inhibitors

In order for the inhibitors of the present invention to be effective, they must reduce the activity of MTHFR in cancer cells to an appropriate extent. As described above, the extent of downregulation of MTHFR activity in response to an inhibitor can be measured in a number of ways. For example, by measuring the cellular level of mRNA or protein, by directly measuring the activity of the MTHFR protein, or by measuring increases in cellular homocysteine, or decreases in cellular 5-methyltetrahydrofolate or methionine, following treatment with a candidate inhibitor and comparing the results to those obtained in the absence of the candidate inhibitor.

Typically, an effective inhibitor will lower the level of MTHFR mRNA, protein, or enzymatic activity, or the level of 5-methyltetrahydrofolate or methionine in cells that have been administered with an MTHFR inhibitor by at least 20% when compared to the corresponding level in the absence of the inhibitor. More typically, the level will be lowered by at least 40%, frequently by at least 60%, by 80%, or by 90% and occasionally by at least 95%. When the level of cellular homocysteine is measured as an indicator of the effectiveness of the inhibitor, this level is typically at least 20% greater than in the absence of the inhibitor. More typically, the level will be increased by at least 40%, frequently by at least 60%, by 80%, or by 90%. The present invention also provides for inhibitors that result in levels of cellular homocysteine as much as 100%, 200% or even 500% greater than in the absence of the inhibitor.

Alternatively, the level of MTHFR mRNA, protein, or enzymatic activity in the presence of an inhibitor can be compared to the level in control cells that do not express functional MTHFR, such as cells homozygous for an MTHFR nonsense mutation. In this case, the level is typically equal to or less than 20-fold, more typically 5-fold and frequently 2-fold over the level in the control cell.

Efficacy of the MTHFR Inhibitors

1. Testing the Effect of MTHFR Inhibitors on Cancer Cells in vitro

The ability of the inhibitors of the present invention to selectively inhibit the growth of cancer cells can be determined by treating a suitable cancer cell-line with a candidate inhibitor and comparing the growth and/or survival of cells thus treated with an appropriate control. In order to determine the selectivity of the inhibitors, an untransformed cell-line can be treated with the inhibitor and monitored for growth and/or survival in a similar manner.

For example, the ability of the MTHFR inhibitors to inhibit proliferation of cancer cells can be assessed by culturing cells of a cancer cell line of interest in a suitable medium and, after an appropriate incubation time, treating the cells with the MTHFR inhibitor. By way of example, when the inhibitor is an oligonucleotide, the cells can be transfected with the inhibitor in the presence of a commercial lipid carrier such as lipofectamine. After treatment, the cells are incubated for a further period of time, then are counted and the numbers compared to an appropriate control, such as cells treated with a standard chemotherapeutic (positive control) and/or untreated cells (negative control). For oligonucleotide inhibitors, a control scrambled oligonucleotide or an oligonucleotide having an unrelated sequence can be used as a negative control in place of, or in addition to, untreated cells.

Alternatively, the MTHFR inhibitors can be tested in vitro by determining their ability to inhibit anchorage-independent growth of tumor cells. Anchorage-independent growth is known in the art to be a good indicator of tumorigenicity. In general, anchorage-independent growth is assessed by plating cells from a selected cancer cell-line onto soft agar and determining the number of colonies formed after an appropriate incubation period. Growth of cells treated with an MTHFR inhibitor can then be compared with that of control cells (as described above).

Similar methods can be employed to test the efficacy of the inhibitors in combination with one or more standard chemotherapeutic(s). Suitable controls in this case would include cells treated with the inhibitor alone and cells treated with the chemotherapeutic(s) alone.

Examples of suitable cancer cell-lines for testing the effects of the MTHFR inhibitors of the present invention include, but are not limited to, colon carcinoma cell-lines SW40, LOVO, CaCo-2, Colo 320, SW620 and SW1222; neuroblastoma cell-lines BE(2)C and SK-N-F1; breast cancer cell-lines MCF7 and SKBr3; glioma cell-line U87-lacZ; prostate cancer cell-line PC3; and lung carcinoma cell-line A549. Many other suitable cancer cell-lines are commercially available. As indicated above, appropriate controls for these tests include untreated cells, cells treated with a control compound, such as a non-specific inhibitor, or untransformed cells treated with the inhibitor.

Typically, the percent of cancerous cells surviving the treatment is at least 20% lower than the initial number of cancerous cells, as measured using any standard assay, such as those described herein. More typically, the number is at least 40% lower, often at least 60% lower or 80% lower, and occasionally 100% lower. The MTHFR inhibitor of the present invention does not significantly affect non-cancerous cells that are not rapidly proliferating. In one embodiment of the present invention the ratio of percent survival of cancer cells over percent survival of normal cells, where the normal cells are not rapidly proliferating, is less than 1. More typically, this ratio is less than 0.9, 0.8, 0.7 or 0.6.

In accordance with the present invention, when the inhibitor is an antisense oligonucleotide, the number of cancerous cells present after administration of an MTHFR antisense nucleotide is at least 2-fold lower than the number of cancerous cells present after administration of a control oligonucleotide that has a polynucleotide sequence less than 70% identical to the reverse complement of a region of an MTHFR nucleic acid. More typically, the number is at least 5-fold greater, frequently at least 10-fold greater, 20-fold greater and occasionally 50-fold greater.

In one embodiment of the present invention, the effect of an antisense oligonucleotide inhibitor is determined by transfecting cancer cells with an inhibitor antisense oligonucleotide or a control oligonucleotide. The initial number of cells is determined, for example using a hemocytometer, and the number of cells surviving treatment is determined, for example using a colorimetric cell protein assay. The percentage of cells surviving the treatment can then be calculated. In a related embodiment, the specificity of the antisense oligonucleotide inhibitor in decreasing the growth of cancer cells only is measured by transfecting a fibroblast (i.e. untransformed) cell-line and determining cell survival as described above.

2. Testing the Effect of MTHFR Inhibitors on Tumors in vivo

The ability of the inhibitors of the invention to inhibit tumor growth or proliferation in vivo can be determined in an appropriate animal model using standard techniques known in the art (see, for example, Enna, et al., Current Protocols in Pharmacology, J. Wiley & Sons, Inc., New York, N.Y.).

In general, current animal models for screening anti-tumor compounds are xenograft models, in which a human tumor has been implanted into an animal. Examples of xenograft models of human cancer include, but are not limited to, human solid tumor xenografts in mice, implanted by sub-cutaneous injection, human solid tumor isografts in mice, implanted by fat pad injection and human solid tumor orthotopic xenografts, all of which can be used in tumor growth assays; experimental models of lymphoma and leukaemia in mice, used in survival assays, and experimental models of lung metastasis in mice.

For example, the inhibitors can be tested in vivo on solid tumors using mice that are subcutaneously grafted bilaterally with 30 to 60 mg of a tumor fragment, or implanted with an appropriate number of cancer cells, on day 0. The animals bearing tumors are mixed before being subjected to the various treatments and controls. In the case of treatment of advanced tumors, tumors are allowed to develop to the desired size, animals having insufficiently developed tumors being eliminated. The selected animals are distributed at random to undergo the treatments and controls. Animals not bearing tumors may also be subjected to the same treatments as the tumor-bearing animals in order to be able to dissociate the toxic effect from the specific effect on the tumor. Chemotherapy generally begins from 3 to 22 days after grafting, depending on the type of tumor, and the animals are observed every day. The inhibitors of the present invention can be administered to the animals, for example, by i.p. injection or bolus infusion. The different animal groups are weighed about 3 or 4 times a week until the maximum weight loss is attained, after which the groups are weighed at least once a week until the end of the trial.

The tumors are measured after a pre-determined time period, or they can be monitored continuously by measuring about 2 or 3 times a week until the tumor reaches a pre-determined size and/or weight, or until the animal dies if this occurs before the tumor reaches the pre-determined size/weight. The animals are then sacrificed and the tissue histology, size and/or proliferation of the tumor assessed.

For the study of the effect of the inhibitors on leukaemias, the animals are grafted with a particular number of cells, and the anti-tumor activity is determined by the increase in the survival time of the treated mice relative to the controls.

To study the effect of the inhibitors of the present invention on tumor metastasis, tumor cells are typically treated with the composition ex vivo and then injected into a suitable test animal. The spread of the tumor cells from the site of injection is then monitored over a suitable period of time.

Similar methods can be employed to test the efficacy of the inhibitors in combination with chemotherapeutic(s). Suitable controls in this case would include cells treated with the inhibitor alone and cells treated with the chemotherapeutic(s) alone.

In vivo toxic effects of the inhibitors can be evaluated by measuring their effect on animal body weight during treatment and by performing haematological profiles and liver enzyme analysis after the animal has been sacrificed.

Non-limiting examples of suitable xenograft models are provided in Table 1.

TABLE 1
Examples of Xenograft Models of Human Cancer

| Cancer Model | Cell Type |
|---|---|
| Tumor Growth Assay | Prostate (PC-3, DU145) |
| Human solid tumor | Breast (MDA-MB-231, MVB-9) |
| xenografts in mice (sub- | Colon (HT-29, SW620) |
| cutaneous injection) | Lung (NCI-H460, NCI-H209, A549) |
| | Pancreatic (ASPC-1, SU86.86) |
| | Pancreatic: drug resistant (BxPC-3) |
| | Skin(A2058, C8161) |
| | Cervical (SIHA, HeLa-S3) |
| | Cervical: drug resistant |
| | (HeLa S3-HU-resistance) |
| | Liver (HepG2) |
| | Brain (U87-MG) |
| | Renal (Caki-1, A498) |
| | Ovary (SK-OV-3) |
| Tumor Growth Assay | Breast: drug resistant |
| Human solid tumor | (MDA-CDDP-S4, MDA- |
| isografts in mice | MB435-To.l)s |
| (fat pad injection) | |
| Survival Assay | Human: Burkitts lymphoma |
| Experimental model | (Non-Hodgkin's) |
| of lymphoma and | (raji) |
| leukaemia in mice | Murine: erythroleukemia |
| | (CB7 Friend |
| | retrovirus-induced) |
| Experimental model of | Human: melanoma (C8161) |
| lung metastasis in mice | Murine: fibrosarcoma (R3) |

Administration of the MTHFR Inhibitors

The inhibitors of the present invention can be administered alone, or in the form of a pharmaceutical composition. The present invention, therefore, provides pharmaceutical compositions comprising one or more MTHFR inhibitors and a pharmaceutically acceptable diluent or excipient. In the case of the pharmaceutical compositions that comprise an inhibitor according to the present invention that is an antisense oligonucleotide, the antisense oligonucleotide may be present as a vector encoding the antisense oligonucleotide. Similarly, in the case where the pharmaceutical composition comprises an inhibitor according to the present invention that is a proteinaceous molecule (i.e. an MTHFR fragment, an MTHFR mutant, an MTHFR specific antibody or a fragment thereof) the molecule may be present as a nucleic acid that encodes the molecule, or as a vector comprising the nucleic acid sequence.

The inhibitors of the present invention and pharmaceutical compositions comprising the inhibitors may be administered in a number of ways depending upon whether local or systemic treatment is desired and upon the area to be treated. Administration may be topical (including ophthalmic and to mucous membranes including vaginal and rectal delivery), pulmonary, e.g. by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g. intrathecal or intraventricular, administration.

The inhibitors of the present invention may be delivered alone or in combination, and may be delivered along with a pharmaceutically acceptable vehicle. Ideally, such a vehicle would enhance the stability and/or delivery properties. The present invention also provides for administration of the inhibitors or pharmaceutical compositions comprising the inhibitors using a suitable vehicle, such as a liposome, microparticle or microcapsule. In various embodiments of the invention, the use of such vehicles may be beneficial in achieving sustained release of the active component.

For administration to an individual for the treatment of cancer, the present invention also contemplates the formulation of the inhibitors or pharmaceutical compositions comprising the inhibitors into oral dosage forms such as tablets, capsules and the like. For this purpose, the inhibitors or pharmaceutical compositions comprising the inhibitors can be combined with conventional carriers, such as magnesium carbonate, magnesium stearate, talc, sugar, lactose, pectin, dextrin, starch, gelatin, tragacanth, methylcellulose, sodium carboxymethyl-cellulose, low melting wax, cocoa butter and the like. Diluents, flavoring agents, solubilizers, lubricants, suspending agents, binders, tablet-disintegrating agents and the like can also be employed, if required. The inhibitors or pharmaceutical compositions comprising the inhibitors can be encapsulated with or without other carriers. In all cases, the proportion of active ingredients in any solid and liquid composition will be at least sufficient to impart the desired activity to the individual being treated upon oral administration. The present invention further contemplates parenteral injection of the inhibitors or pharmaceutical compositions comprising the inhibitors, in which case they are used in the form of a sterile solution containing other solutes, for example, enough saline or glucose to make the solution isotonic.

For administration by inhalation or insufflation, the inhibitors or pharmaceutical compositions comprising the inhibitors can be formulated into an aqueous or partially aqueous solution, which can then be utilized in the form of an aerosol. The present invention also contemplates topical use of the inhibitors or pharmaceutical compositions comprising the inhibitors. For this purpose they can be formulated as dusting powders, creams or lotions in pharmaceutically acceptable vehicles, which are applied to affected portions of the skin.

The present invention also provides for administration of antisense oligonucleotide, protein and peptide inhibitors in the form of a genetic vector construct that is designed to direct the in vivo synthesis of the inhibitor. Suitable vectors include viral vectors, such as an adenoviral, adeno-associated viral, retroviral, lentiviral, baculovirus, or herpes viral vectors. Within the vector construct, the nucleic acid sequence encoding the inhibitor is under the control of a suitable promoter. As described herein, the vector construct may additionally contain other regulatory control elements to provide efficient transcription and/or translation of the nucleic acid encoding the inhibitor.

The preparation of a vector comprising a nucleic acid sequence encoding and antisense oligonucleotide according to the present invention has been described herein. A worker skilled in the art would readily appreciate that a vector comprising the coding sequence for a proteinaceous inhibitor according to the present invention can be prepare using the same standard techniques.

Methods of constructing and administering such genetic vector constructs for the in vivo synthesis of antisense oligonucleotides, proteins or peptides are well-known in the art. For example, see Ausubel, et al., (2000) Current Protocols in Molecular Biology, Wiley & Sons, New York, N.Y. An efficient method for the introduction, expression and accumulation of antisense oligonucleotides in the cell nucleus is described in U.S. Pat. No. 6,265,167. This method allows the antisense oligonucleotide to hybridize to the sense mRNA in the nucleus, and thereby prevents the antisense oligonucleotide being either processed or transported into the cytoplasm.

The dosage requirements for the inhibitors of the present invention or pharmaceutical compositions comprising the inhibitors vary with the particular compositions employed, the route of administration, the severity of the symptoms presented and the particular subject being treated. Dosage requirements can be determined by standard clinical techniques known to a worker skilled in the art. Treatment will generally be initiated with small dosages less than the optimum dose of the compound. Thereafter the dosage is increased until the optimum effect under the circumstances is reached. In general, the inhibitors or pharmaceutical compositions comprising the inhibitors are administered at a concentration that will generally afford effective results without causing any harmful or deleterious side effects. Administration can be either as a single unit dose or, if desired, the dosage can be divided into convenient subunits that are administered at suitable times throughout the day.

Applications

The present invention provides MTHFR inhibitors that selectively decrease the growth of cancer cells, while leaving non-cancerous cells fully or partly unaffected. The inhibitors of the present invention, therefore, can be used to treat, stabilize or prevent cancer. In this context, the inhibitors exert cytotoxic or cytostatic effects that cause a reduction in the size of a tumor, slow or prevent an increase in the size of a tumor, increase the disease-free survival time between the disappearance of a tumor and its reappearance, prevent an initial or subsequent occurrence of a tumor (e.g. metastasis), increase the time to progression, reduce one or more adverse symptom associated with a tumor, or increase the overall survival time of a subject having cancer.

In accordance with the present invention the one or more MTHFR inhibitors or pharmaceutical compositions comprising the one or more MTHFR inhibitors is used to selectively inhibit cancer cells in vitro or in vivo, while leaving normal cells fully or partially unaffected. A further embodiment of the present invention provides a method for treating a mammal suffering from cancer by administering one or more MTHFR inhibitors or a pharmaceutical composition comprising one or more MTHFR inhibitors. In a related embodiment the MTHFR inhibitor or pharmaceutical compositions is used to selectively inhibit the growth and/or metastasis of cancer cells in vitro or in vivo in a mammal in need of such therapy. In a specific embodiment of the present invention the mammal is a human.

Examples of cancers which may be may be treated or stabilized in accordance with the present invention include, but are not limited to leukaemias, carcinomas, adenocarcinomas, melanomas and sarcomas. Carcinomas, adenocarcinomas and sarcomas are also frequently referred to as “solid tumors.” Examples of commonly occurring solid tumors include, but are not limited to, cancer of the brain, breast, cervix, colon, head and neck, kidney, lung, ovary, pancreas, prostate, stomach and uterus, as well as non-small cell lung cancer and colorectal cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease—acute or chronic; (2) the type of cell involved; myeloid (myelogenous), lymphoid (lymphogenous), or monocytic; and (3) the increase or non-increase in the number of abnormal cells in the blood—leukemic or aleukemic (subleukemic). Leukemia includes, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia.

The term “sarcoma” generally refers to a tumor which is made up of a substance like the embryonic connective tissue and is generally composed of closely packed cells embedded in a fibrillar or homogeneous substance. Sarcomas include chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

The term “melanoma” is taken to mean a tumor arising from the melanocytic system of the skin and other organs. Melanomas include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, and superficial spreading melanoma.

The term “carcinoma” refers to a malignant new growth made up of epithelial cells tending to infiltrate the surrounding tissues and give rise to metastases. Exemplary carcinomas include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

The term “carcinoma” also encompasses adenocarcinomas. Adenocarcinomas are carcinomas that originate in cells that make organs which have glandular (secretory) properties or that originate in cells that line hollow viscera, such as the gastrointestinal tract or bronchial epithelia. Examples include, but are not limited to, adenocarcinomas of the breast, lung, pancreas, colon and prostate.

Additional cancers encompassed by the present invention include, for example, Hodgkin's Disease, Non-Hodgkin's lymphoma, multiple myeloma, neuroblastoma, rhabdomyosarcoma, primary thrombocytosis, primary macroglobulinemia, small-cell lung tumors, primary brain tumors, gliomas, malignant pancreatic insulanoma, malignant carcinoid, urinary bladder cancer, premalignant skin lesions, gliomas, testicular cancer, thyroid cancer, esophageal cancer, genitourinary tract cancer, malignant hypercalcemia, endometrial cancer, adrenal cortical cancer, mesothelioma and medulloblastoma.

In one embodiment of the present invention, the MTHFR inhibitors are administered to a subject in order to treat, stabilize, or prevent a solid tumor. In another embodiment, the MTHFR inhibitors are used to treat, stabilize, or prevent a carcinoma. In a further embodiment, the MTHFR inhibitors are used to treat, stabilize, or prevent an adenocarcinoma. In a specific embodiment, the MTHFR inhibitors are used to treat, stabilize, or prevent breast cancer, colon cancer, colorectal cancer, lung cancer, prostate cancer, cancer of the nervous system or brain cancer.

Combination Therapy

For the treatment, stabilization or prevention of cancer, one or more MTHFR inhibitor of the invention can be administered to a subject alone or in combination with one or more anti-cancer therapeutics. The one or more MTHFR inhibitor can be administered before, during or after treatment with the anti-cancer therapeutic. An “anti-cancer therapeutic” in the context of the present invention is a compound, composition or treatment that prevents or delays the growth and/or metastasis of cancer cells. Such anti-cancer therapeutics include, but are not limited to, chemotherapeutic drugs, radiation, gene therapy, hormonal manipulation, immunotherapy and antisense oligonucleotide therapy. It is to be understood that anti-cancer therapeutics suitable for use in the present invention also include novel compounds or treatments developed in the future.

When the inhibitors of the present invention are used in combination with one or more chemotherapeutic drug, the chemotherapeutic drug(s) can be selected from a wide range of anti-cancer drugs known in the art. Known chemotherapeutic drugs include those that are applicable to a range of cancers, such as doxorubicin, capecitabine, mitoxantrone, irinotecan (CPT-11), cisplatin, 5-fluorouracil (5-FU) and gemcitabine, as well as those that are specific for the treatment of a particular type of cancer. The present invention contemplates the use of both types of chemotherapeutic agent in conjunction with the inhibitors of the present invention. Exemplary chemotherapeutics that can be used alone or in various combinations for the treatment specific cancers are provided in Table 2. One skilled in the art will appreciate that many other chemotherapeutics are available and that the following list is representative only.

TABLE 2
Exemplary Chemotherapeutics Used in the Treatment of Some Common Cancers

| CANCER | CHEMOTHERAPEUTIC | |
|---|---|---|
| Acute lymphocytic | Pegaspargase (e.g. Oncaspar ®) | L-asparaginase |
| leukaemia (ALL) | Interleukin-2 (e.g. Proleukin ®) | Cytarabine |
| Acute myeloid | Cytarabine | Idarubicin |
| leukaemia (AML) | | |
| Brain cancer | Procarbazine (e.g. Matulane ®) | Nitrosoureas |
| | Platinum analogues | Temozolomide |
| Breast cancer | Capecitabine (e.g. Xeloda ®) | Cyclophosphamide |
| | 5-fluorouracil (5-FU) | Carboplatin |
| | Paclitaxel (e.g. Taxol ®) | Cisplatin |
| | Docetaxel (e.g. Taxotere ®) | Ifosfamide |
| | Epi-doxorubicin (epirubicin) | Doxorubicin (e.g. Adriamycin ®) |
| | Trastuzumab (Herceptin ®) | Tamoxifen |
| Chronic myeloid | Low-dose Interferon (IFN)-alpha | |
| leukaemia (CML) | Cytarabine | |
| Colon cancer | Edatrexate (10-ethyl-10-deaza-aminopterin) | |
| | Methyl-chloroethyl-cyclohexyl-nitrosourea | |
| | 5-fluorouracil (5-FU) | Levamisole |
| | Fluorodeoxyuridine (FUdR) | Vincristine |
| | Capecitabine (e.g. Xeloda ®) | Oxaliplatin |
| Colorectal cancer | Irinotecan (CPT-11, e.g. Camptosar ®) | |
| | Loperamide (e.g. Imodium ®) | Levamisole |
| | Topotecan (e.g. Hycamtin ®) | Methotrexate |
| | Capecitabine (e.g. Xeloda ®) | Oxaliplatin |
| | 5-fluorouracil (5-FU) | |
| Gall bladder | 5-fluorouracil (5-FU) | |
| Genitourinary | Docetaxel (e.g. Taxotere ®) | |
| cancer | | |
| Head and neck | Docetaxel (e.g. Taxotere ®) | Cisplatin |
| cancer | | |
| Non-Hodgkin's | Procarbazine (e.g. Matulane ®) | Cytarabine |
| Lymphoma | Rituximab (e.g. Rituxan ®) | Etoposide |
| Non-small-cell lung | Vinorelbine Tartrate (e.g. Navelbine ®) | |
| (NSCL) cancer | Irinotecan (CPT-11, e.g. Camptosar ®) | |
| | Docetaxel (e.g. Taxotere ®) | Paclitaxel (e.g. Taxol ®) |
| | Gemcitabine (e.g. Gemzar ®) | Topotecan |
| Oesophageal cancer | Porfimer Sodium (e.g. Photofrin ®) | |
| | Cisplatin | |
| Ovarian cancer | Irinotecan (CPT-11, e.g. Camptosar ®) | |
| | Topotecan (e.g. Hycamtin ®) | Paclitaxel (e.g. Taxol ®) |
| | Docetaxel (e.g. Taxotere ®) | Amifostine (e.g. Ethyol ®) |
| | Gemcitabine (e.g. Gemzar ®) | |
| Pancreatic cancer | Irinotecan (CPT-11, e.g. Camptosar ®) | |
| | Gemcitabine (e.g. Gemzar ®) | 5-fluorouracil (5-FU) |
| Promyelocytic | Tretinoin (e.g. Vesanoid ®) | |
| leukaemia | | |
| Prostate cancer | Goserelin Acetate (e.g. Zoladex ®) | Liarozole |
| | Mitoxantrone (e.g. Novantrone ®) | Flutamide (e.g. Eulexin ®) |
| | Prednisone (e.g. Deltasone ®) | Terazosin (e.g. Hytrin ®) |
| | Nilutamide (e.g. Nilandron ®) | Cyclophosphamide |
| | Finasteride (e.g. Proscar ®) | Estramustine |
| | Doxazosin (e.g. Cardura ®) | |
| | Docetaxel (e.g. Taxotere ®) | |
| | Luteinizing hormone releasing hormone agonist | |
| Renal cancer | Capecitabine (e.g. Xeloda ®) | |
| | Gemcitabine (e.g. Gemzar ®) | |
| | Interleukin-2 (e.g. Proleukin ®) | |
| Small cell lung | Cyclophosphamide | Vincristine |
| cancer | Doxorubicin | Etoposide |
| Solid tumors | Gemicitabine (e.g. Gemzar ®) | Cyclophosphamide |
| | Capecitabine (e.g. Xeloda ®) | Ifosfamide |
| | Paclitaxel (e.g. Taxol ®) | Cisplatin |
| | Docetaxel (e.g. Taxotere ®) | Carboplatin |
| | Epi-doxorubicin (epirubicin) | Doxorubicin (e.g. Adriamycin ®) |
| | 5-fluorouracil (5-FU) | |

In one embodiment of the invention, the MTHFR inhibitor is administered in combination with one or more generally applicable chemotherapeutic, such as doxorubicin, capecitabine, mitoxantrone, irinotecan (CPT-11), cisplatin, 5-fluorouracil (5-FU) or gemcitabine. In another embodiment, the MTHFR inhibitor is administered in combination with one or more specific chemotherapeutic, such as docetaxel or paclitaxel.

As indicated above, combinations of chemotherapeutics may be employed. Combination therapies using standard cancer chemotherapeutics are well known in the art and such combinations also can be used in conjunction with the inhibitors of the invention.

Exemplary combination therapies include for the treatment of breast cancers the combination of epirubicin with paclitaxel or docetaxel, or the combination of doxorubicin or epirubicin with cyclophosphamide. Polychemotherapeutic regimens are also useful and may consist, for example, of doxorubicin/cyclophosphamide/5-fluorouracil or cyclophosphamide/epirubicin/5-fluorouracil. Many of the above combinations are useful in the treatment of a variety of other solid tumors.

Combinations of etoposide with either cisplatin or carboplatin are used in the treatment of small cell lung cancer. In the treatment of stomach or oesophageal cancer, combinations of doxorubicin or epirubicin with cisplatin and 5-fluorouracil are useful. For colorectal cancer, CPT-11 in combination with 5-fluorouracil-based drugs, or oxaliplatin in combination with 5-fluorouracil-based drugs can be used. Oxaliplatin may also be used in combination with capecitabine.

Other examples include the combination of cyclophosphamide, doxorubicin, vincristine and prednisone in the treatment of non-Hodgkin's lymphoma; the combination of doxorubicin, bleomycin, vinblastine and dacarbazine (DTIC) in the treatment of Hodgkin's disease and the combination of cisplatin or carboplatin with any one, or a combination, of gemcitabine, paclitaxel, docetaxel, vinorelbine or etoposide in the treatment of non-small cell lung cancer.

Various sarcomas are treated by combination therapy, for example, for osteosarcoma combinations of doxorubicin and cisplatin or methotrexate with leucovorin are used; for advanced sarcomas etoposide can be used in combination with ifosfamide; for soft tissue sarcoma doxorubicin or dacarbazine can be used alone or, for advanced sarcomas doxorubicin can be used in combination with ifosfamide or dacarbazine, or etoposide in combination with ifosfamide.

Ewing's sarcoma/peripheral neuroectodermal tumor (PNET) or rhabdomyosarcoma can be treated using etoposide and ifosfamide, or a combination of vincristine, doxorubicin and cyclophosphamide.

The alkylating agents cyclophosphamide, cisplatin and melphalan are also often used in combination therapies with other chemotherapeutics in the treatment of various cancers.

The present invention also contemplates the use of the MTHFR inhibitors as “sensitizing agents,” which selectively inhibit the growth of cancer cells. In this case, the inhibitor alone does not have a cytotoxic effect on the cell, but selectively arrests or slows the growth of cancer cells. The inhibitor thus provides a means of weakening the cancer cells, and thereby facilitates the benefit from conventional anti-cancer therapeutics.

Kits

The present invention additionally provides for therapeutic kits containing the inhibitors in pharmaceutical compositions for use in the treatment of cancer. The contents of the kit can be lyophilized and the kit can additionally contain a suitable solvent for reconstitution of the lyophilized components. The kit may further comprise one or more standard chemotherapeutic for use in combination with the MTHFR inhibitor(s). Individual components of the kit would be packaged in separate containers and, associated with such containers, can be a notice in the form prescribed by a governmental agency regulating the manufacture, use or sale of pharmaceuticals or biological products, which notice reflects approval by the agency of manufacture, use or sale for human administration.

When the components of the kit are provided in one or more liquid solutions, the liquid solution can be an aqueous solution, for example a sterile aqueous solution. For in vivo use, the expression construct may be formulated into a pharmaceutically acceptable syringeable composition. In this case the container means may itself be an inhalant, syringe, pipette, eye dropper, or other such like apparatus, from which the formulation may be applied to an infected area of the animal, such as the lungs, injected into an animal, or even applied to and mixed with the other components of the kit.

The components of the kit may also be provided in dried or lyophilized forms. When reagents or components are provided as a dried form, reconstitution generally is by the addition of a suitable solvent. It is envisioned that the solvent also may be provided in another container means. Irrespective of the number or type of containers, the kits of the invention also may comprise, or be packaged with, an instrument for assisting with the injection/administration or placement of the ultimate complex composition within the body of an animal. Such an instrument may be an inhalant, syringe, pipette, forceps, measured spoon, eye dropper or any such medically approved delivery vehicle.

To gain a better understanding of the invention described herein, the following examples are set forth. It should be understood that these examples are for illustrative purposes only. Therefore, they should not limit the scope of this invention in any way.

### EXAMPLES

### General

Cell Lines

Human fibroblasts MCH 51, MCH 75 and WG 1554 were obtained from the Repository for Mutant Human Cell Strains (Montreal Children's Hospital, Montreal, Canada). WG 1554 is homozygous for a nonsense mutation in MTHFR (Goyette et al, (1994) Nature Genetics, 7:175-200). Human colon carcinoma cell lines CaCo-2, Colo 320, and SW620 were obtained from American Type Culture Collection (Rockville Md.); human colon carcinoma cell line SW 1222 was a gift from Dr. N. Beauchemin (McGill University, Montreal, Canada). The carcinoma lines were genotyped for the MTHFR variant at bp 677 by PCR amplification and HinfI digestion, as previously reported (Frosst et al, (1995) Nature Genetics, 10:111-113). SW 1222 and CaCo-2 were shown to carry the wild type alanine allele (A) whereas Colo 320 and SW620 carry the mutant valine allele (V). Two neuroblastoma lines, BE(2)C and SK-N-F1, were obtained from the American Type Culture Collection. The breast carcinoma cell lines, MCF7 and SKBr3, were a gift from Dr. Morag Park (McGill University, Montreal, Canada). A549 lung carcinoma cells and PC3 prostate carcinoma cells were obtained from the American Type Culture Collection. The MCF7 cell line was grown in α-MEM (Life Technologies, Rockville Md.) and the SKBr3 cell line was maintained in D-MEM (Life Technologies). Media for both lines was supplemented with 10% fetal bovine serum (Intergen, Purchase N.Y.). A549 and PC3 lines were grown in F12 medium with 10% fetal bovine serum.

All media was also supplemented with 50 IU/ml penicillin (Life Technologies), 50 μg/ml streptomycin (Life Technologies), 0.5 μg/ml fungizone reagent (Life Technologies). All cell lines were cultured in 75 cm² flasks in a humidified 37° C. incubator in 5% CO₂.

Deficient Culture Media

MEM and MEM without folate and methionine supplemented with 100 mM sodium pyruvate (F-M-) were obtained from Life Technologies. For methionine-deficient (M-) media, 2.3 μM folate (Sigma-Aldrich, Oakville ON) was added to the F-M- media. For all media, 5% fetal bovine serum (Intergen), 5% iron enriched calf serum (Intergen), 50 IU/ml penicillin (Life Technologies), 50 μg/ml streptomycin (Life Technologies), and 0.5 μg/ml fungizone reagent (Life Technologies) were added. For M-H+ media, 0.44 mM homocysteine (Sigma-Aldrich) and 1.5 μM vitamin B₁₂ (Sigma-Aldrich) was added to the M- media. Dialyzed serum was used for all deficient media.

Cell Survival Studies of Cells in Deficient Media

Cell viability studies were performed in 6-well tissue culture plates starting with 30,000-50,000 cells per well and 3 replicates for each condition. The initial number of cells were estimated with a hemacytometer. Cell survival in MEM was used as a control for proliferation in deficient media (M-, M-H+). Surviving cells were counted using the FluoroReporter Colorimetric Cell Protein Assay Kit (Molecular Probes, Eugene Oreg.).

Oligonucleotides

For assays to determine the effect of an MTHFR antisense oligonucleotide on cell viability, the following antisense oligonucleotides were used:
1. EX5 (phosphorothioate 5′-AGCTGCCGAAGGGAGTGGTA-3′) [SEQ ID NO:16]—designed to bind exon 5 of the MTHFR gene
2. CT 677, (phosphorothioate 5′-TGCTGTCGGAGCGATAGGTC-3′) [SEQ ID NO:18]—a control oligonucleotide with six base pair mismatches
3. CTSEX5, (phosphorothioate 5′-GTGACGTAGGACAGCGATGG-3′) [SEQ ID NO:17]—a control oligonucleotide with a scrambled sequence
4. Oligonucleotide SEQ ID NO:19 (phosphorothioate 5′-AGCTGCcGAAGGGAGTGGTA-3′, lowercase “c” indicates a methylated cytosine)—designed to bind exon 5 of the MTHFR gene
5. Oligonucleotide SEQ ID NO:20 (phosphorothioate 5′-GTGGACTAGGACAGcGATGG-3′, lowercase “c” indicates a methylated cytosine)—a control oligonucleotide with a scrambled sequence

The above oligonucleotides were synthesized using standard solid-phase DNA synthesis procedures. This region of exon 5 was chosen because a BLAST search of the human expressed sequence (EST) database indicated that this sequence did not have significant identity to any other reported EST in humans. In addition, no sequence variations have been reported for this MTHFR exon, suggesting that an antisense oligonucleotide to exon 5 may bind all MTHFR alleles. The sequences of CT677 and CTSEX5 did not show homology to any known human genes in a BLAST search. These oligonucleotides were synthesized as phosphorothioate oligonucleotides in which one of the non-bridging phosphoryl oxygens of each nucleotide was replaced with sulphur. This modification dramatically improves nuclease stability and pharmacokinetics in vitro and in vivo.

Transfection with Oligonucleotides and Cell Counting

Cells were plated in 6-well dishes at 50-70% confluence and incubated overnight in complete medium (Life Technologies). Each well was washed once with OPTI-MEM I (Life Technologies). The cells were then overlayed with 1 ml of Opti-MEM I media containing 12 μg/ml Lipofectin reagent (Life Technologies) per 400 nM of oligonucleotide. The media was replaced with complete media (2-4 ml) after 5 hour incubation at 37° C. with the antisense oligonucleotides. Transfection with oligonucleotides was performed on 3 consecutive days followed by a 3-day period of regrowth in MEM. Cells were counted by SRB staining as outlined in the FluoroReporter Colorimetric Cell Protein Assay Kit (Molecular Probes). In each experiment, treatments were performed in triplicate.

In dose response experiments, the total oligonucleotide concentration was held constant at 400 nM by supplementing the tested oligonucleotide with the control oligonucleotide (Basilion et al, (1999) Molec. Pharmacol., 56:359-369).

Protein Extraction after Treatment with Oligonucleotides

For Western blot analysis and MTHFR enzyme assays, 6×10⁵ SW620 colon carcinoma cells were plated in 100 mm tissue culture treated petri dishes. After transfection of cells with oligonucleotides, the cells were harvested, and crude protein extracts from cell pellets were obtained by freezing the pellet at −70° C. and thawing to 4° C. three successive times. The cell pellet was then resuspended in 0.1 M KPO₄ pH 6.3 with 2 μg/ml aprotinin (Boehringer Mannheim, Laval, Quebec) and 2 ug/ml leupeptin (Amersham Pharmacia Biotech, Piscataway N.J.). Cellular debris was cleared by centrifugation at 14,000 rpm for 10 min. Protein concentration was assayed using the Bradford method (Bradford, 1976) according to the manufacturer's instructions (BioRad, Mississauga ON).

MTHFR Enzyme Assay

Enzyme activity was measured in the reverse direction, in crude protein extracts, as previously described (Christensen et al, (1997) ARTERIOSCLER. Thromb. Vasc. Biol., 17:569-573). Equal amounts of protein (˜60 μg) were used per assay. Enzyme activity was expressed as nmol formaldehyde formed per mg protein/h.

Western Blot Analysis

Equal amounts of protein (35-60 μg) were loaded onto a 10% SDS polyacrylamide gel. Transfer was performed in a transfer buffer (39 mM glycine, 49 mM Tris base, 0.037% SDS, 20% methanol) for 2-3 hour at 70 V to nitrocellulose (Hybond ECL membrane, Amersham Pharmacia Biotech). The membrane was blocked with 5% non-fat skim milk in PBS-0.5% Tween 20 (Tween 20; BioRad) overnight at 4° C. The MTHFR protein was detected using a rabbit anti-porcine MTHFR antibody at a dilution of 1:1000 in 5% non-fat skim milk in PBS-0.5% Tween 20 incubated for 4 to 6 hour at 4° C. After three successive washes in PBS-0.5% Tween 20, anti-rabbit horseradish peroxidase-conjugated antibody (Amersham Pharmacia Biotech) was used as a secondary antibody. The immunocomplexes were visualized by enhanced chemiluminescence with an ECL kit (Amersham Pharmacia Biotech). Quantitation of protein was determined by scanning the films with a flat-bed scanner (Hewlett Packard Scan). The MTHFR and actin band areas were calculated; MTHFR protein level is expressed as a ratio of MTHFR/actin.

Statistical Analysis

One-way ANOVA was performed using SPSS software, Version 10.0 (SPSS Inc., Chicago Ill.), to analyze cell survival after treatment with EX5. The Student t-test was used to evaluate differences in MTHFR activity, and to analyze cell survival data of fibroblast cell lines treated with EX5 antisense.

### Example 1

### Decreased Cell Viability in Cancer Cells after Transfection of an MTHFR Antisense Oligonucleotide

The following cancer cell lines were used in these experiments: SW620 colon carcinoma (ATCC Accession No. CCL-227), LOVO colon carcinoma (ATCC Accession No. CCL 229), BEC 2 neuroblastoma (ATCC Accession No. CRL 2268), SK-N-F1 neuroblastoma (ATCC Accession No. CRL 2142), MCF7 breast cancer (ATCC Accession No. HTB 2), SKBr3 breast cancer (ATCC Accession No. HTB 30), and U87-lacZ glioma cell lines (Li et al., (1999) Clin. Cancer Res. 5:637-642).

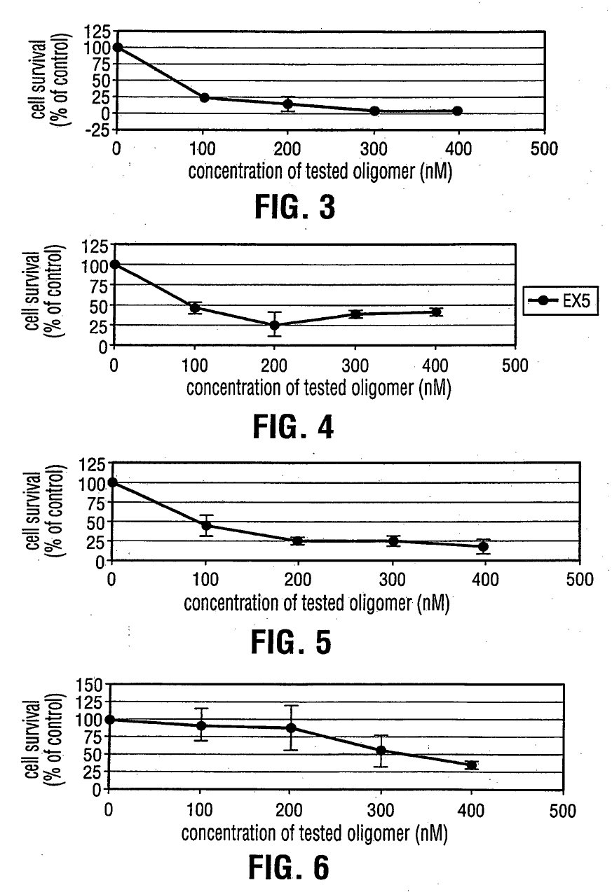

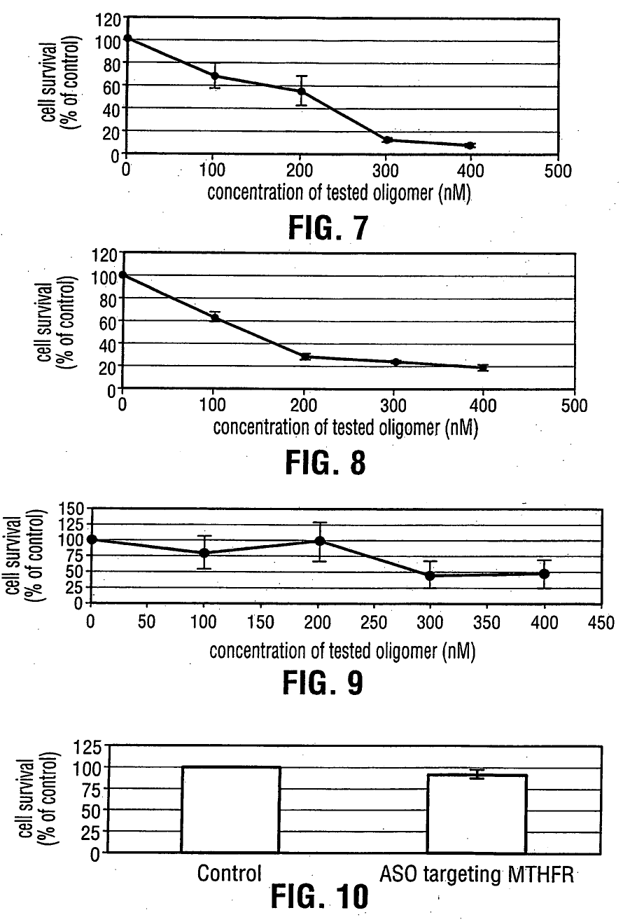

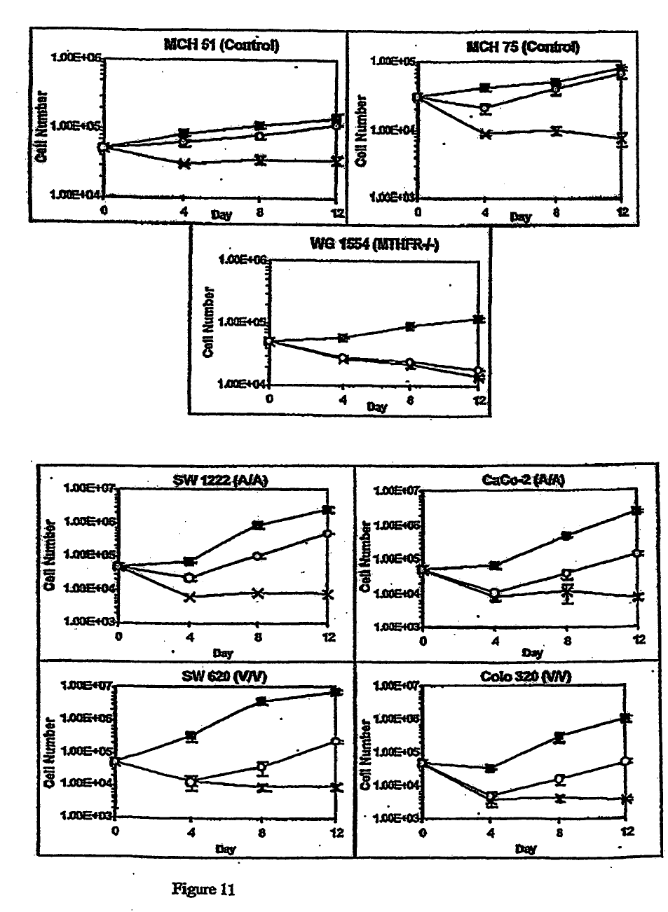

Prior to the transfection of tumor cells with these phosphorothioate oligonucleotides, the cells were plated at 50-60% confluence in 6-well plates or 10 cm tissue culture dishes and incubated overnight at 37° C. and 5% CO₂. The next day, the cells were washed with OPTI-MEM I Reduced Serum Media (Gibco, BRL) and treated with the indicated concentration of the MTHFR antisense oligonucleotide or one of the control oligonucleotides (FIGS. 3-11) and 12 ug/ml Lipofectin Reagent (Gibco-BRL) in OPTI-MEM I Reduced Serum Media. For the U87-lacZ glioma cell line, the control oligonucleotide CT677 was used as the control oligonucleotide; for all the other cell lines, the scrambled oligonucleotide CTSEX5 was used as the control oligonucleotide.

After a five hour incubation at 37° C. and 5% CO₂ to allow transfection of the oligonucleotide into the cells, the OPTI-MEM I Reduced Serum Media containing the oligonucleotide and Lipofectin was replaced with MEM supplemented with 5% FBS and 5% calf serum. The next day the transfection protocol was repeated. For the second transfection, the cells were washed with Opti-MEM I media and incubated for five hours with the MTHFR antisense or control oligonucleotide and Lipofectin reagent, as described above. Then, the media was replaced with MEM supplemented with 5% FBS and 5% calf serum. The following day, a third transfection was performed as described for the second transfection. After this transfection, the cells were allowed to grow in the supplemented MEM media for two to four days. Then, the number of cells attached to the tissue culture dish was determined using a colorimetric cell protein assay kit, according to the manufacturer's protocol (FluoroReporter® Colorimetric Cell Protein Assay Kit F-2961 from Molecular Probes, Eugene Oreg.). This kit contains the anionic xanthene dye, sulforhadamine B, that forms an electrostatically stabilized complex with basic amino acid residues under moderately acidic conditions. This protein-dye complex was detected spectrophotometrically after removal of unbound dye from TCA-fixed cells.

### Example 2

### Failure of an MTHFR Antisense Oligonucleotide to Reduce Cell Viability of Non-Cancerous Cells

To determine the effect of an MTHFR antisense oligonucleotide on non-cancerous cells, the human diploid fibroblast cell line WG1554, which carries 2 nonsense mutations for MTHFR and thus does not produce functional MTHFR protein, was also tested in the above transfection assay (Goyette et al., (1994) Nat. Genet.7:195-200). In particular, the cells were subjected to three rounds of transfection with 400 nM of the antisense oligonucleotide designed to bind exon 5 of MTHFR or the control oligonucleotide and allowed to recover for three days. In contrast to the previous results with tumor cell lines, there was no difference in cell survival between WG1554 cells treated with the MTHFR antisense or control oligonucleotide (FIG. 10). This result indicates that the MTHFR antisense oligonucleotide is more toxic to cancerous cells than non-cancerous cells that do not express MTHFR. Thus, MTHFR antisense oligonucleotides may produce few adverse side-effects if administered to human subjects.

### Example 3

### MTHFR Antisense Oligonucleotides Decrease the Cell Viability of Methionine-Dependent Transformed Cells

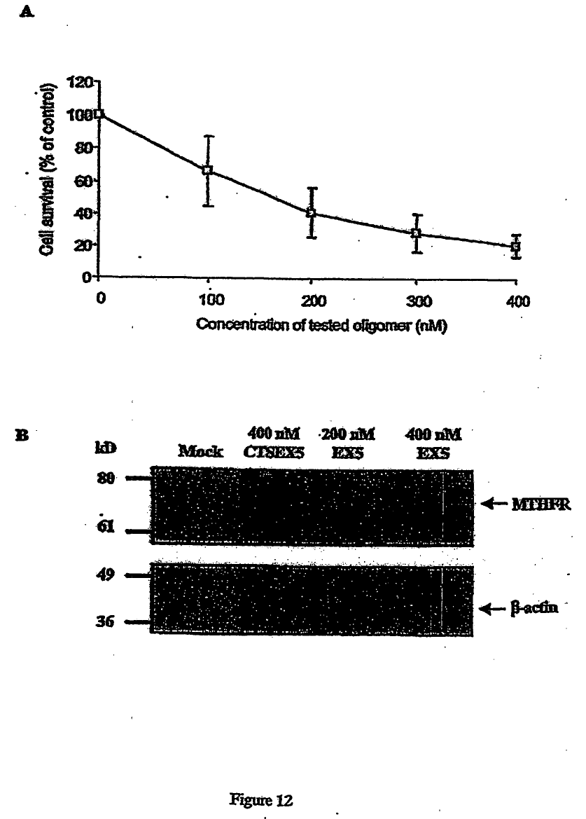

Three fibroblast strains (FIG. 11) and 4 colon carcinoma lines (FIG. 12) were grown in MEM, MEM without methionine (M-), or MEM without methionine supplemented with homocysteine and vitamin B₁₂ (M-H+). The latter medium served to examine de novo synthesis of methionine from homocysteine and 5-methyltetrahydrofolate, catalyzed by vitamin B₁₂-dependent methionine synthase. 5-Methyltetrahydrofolate is the product of the MTHFR reaction. All seven lines showed sensitivity to the M- medium; growth was significantly reduced in this medium compared to that in MEM. Control fibroblasts (MCH 51, MCH 75) could maintain virtually normal growth in the M-H+ medium. However, the fibroblast strain WG 1554, which is homozygous for a nonsense mutation in MTHFR (Goyette et al, 1994), was unable to restore growth in the M-H+ medium. The carcinoma lines cultured in the M-H+ medium increased their proliferation only slightly through endogenous methionine synthesis (FIG. 12). The cell numbers were just a small percentage (5%-20%) of the values obtained in MEM. These carcinoma lines are not compromised with respect to MTHFR activity, although two of the lines (Colo 320 and SW620) have the valine allele, which is associated with mild enzymatic deficiency.

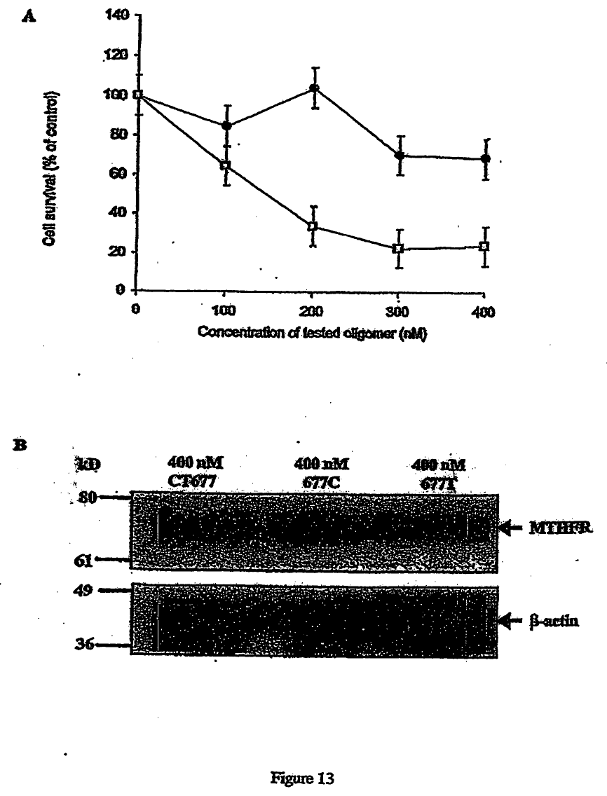

FIG. 13A demonstrates a dose-dependent decrease in cell survival (p<0.01, one-way ANOVA) after treatment of SW620 carcinoma cells with the MTHFR antisense oligonucleotide EX5. At the maximal dose of 400 nM, cell survival decreased approximately 80% compared to that of cells treated with the scrambled control oligonucleotide CTSEX5.

To ensure that MTHFR expression was altered, Western blotting was used to analyze immunoreactive MTHFR protein, after three consecutive treatments with the EX5 antisense oligonucleotide. FIG. 13B demonstrates a significant decrease in MTHFR protein levels after EX5 treatment, compared to treatment with the scrambled control, CTSEX5, or compared to treatment with Lipofectin reagent only (mock transfection). After normalization to actin, MTHFR protein levels following treatment with the control oligo were 94% of mock-treated cells, whereas treatment with 200 nM and 400 nM of EX5, MTHFR protein levels were 39% and 25%, respectively, of that in mock-treated cells (average of 3 Western blots).

### Example 4

### Cell Survival of Normal Human Fibroblasts, Breast Carcinoma Cells and Neuroblastoma Lines After Treatment With 400 nM of EX5

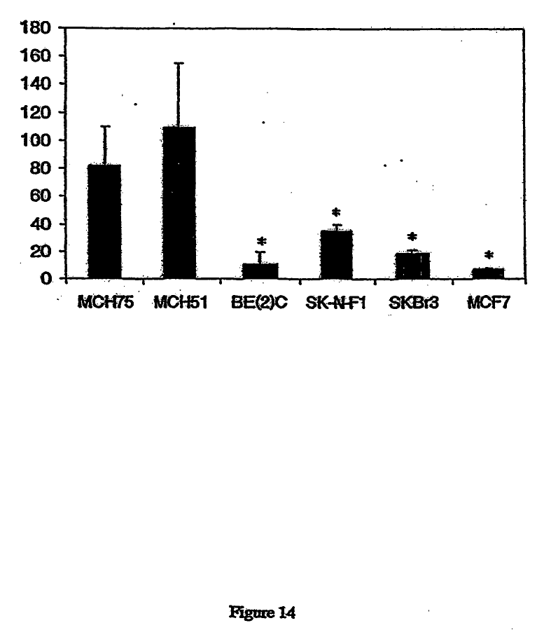

After treatment with 400 nM of EX5 as described above, two neuroblastoma cell lines (BE(2)C and SK-N-F1) showed significant decreases in cell survival compared to control antisense oligonucleotide treated cells: decreases of 80% (p<0.001) and 65% (p<0.01), respectively. Similarly, the breast carcinoma cell line SKBr3 showed a 80% (p<0.0001) decrease in cell survival and the MCF7 breast carcinoma line showed a 92% (p<0.0001) reduction in cell survival compared to control oligonucleotide CTSEX5 treated cells. Contrary to data obtained in transformed lines, two normal human fibroblast cell lines (MCH 75 and MCH 51) treated with 400 nM of EX5 did not exhibit significant differences in cell survival compared to CTSEX5 treated cells (p>0.05). These results are summarized in FIG. 14.

### Example 5

### In vivo Effects of the Downregulation of MTHFR Expression on Tumors

The Min (multiple intestinal neoplasia) mouse is an established mouse model for colon cancer. It carries a mutation in the APC gene, the same gene that is mutated in human hereditary and sporadic colorectal tumors. These mice develop multiple tumors (from 30 to 100) at several months of age.

Min mice, which carry one copy of the APC mutation, were crossed to MTHFR-deficient mice with a heterozygous knockout of the MTHFR gene. These heterozygous mice carry one copy of the null allele (Mthfr +/−) and, therefore, have 50% of MTHFR activity compared to normal mice.

Min mice (n=20) carrying just the APC mutation had a mean tumor number of 75±6.6 (standard error), whereas the Min mice with the APC mutation as well as a MTHFR null allele had a mean tumor number of 36±2.7. This difference in tumor number is highly significant (p<0.0001). In addition, the sizes of the tumors were smaller in the Min mice carrying the MTHFR mutation (91.9% of tumors were less than 1 mm), compared to Min mice without the MTHFR mutation (76.3% of tumors were less than 1 mm).

Therefore, partial inhibition of MTHFR in transformed cells is associated with decreased numbers of tumors and decreased tumor growth. Partial inhibition of MTHFR in normal cells does not appear to be deleterious, since the mice with a heterozygous knockout of MTHFR (Mthfr +/−), without any other mutations, are similar to the normal mice in appearance, birth weight, growth and survival.

These studies suggest that inhibition of MTHFR may be more deleterious to rapidly-growing cells than to normal cells. This is consistent with studies in cultured cells outlined above, which demonstrate that transformed cells have a higher requirement for methionine than normal cells.

### Example 6

### In Vivo Animal Model for Study of MTHFR Antisense Oligonucleotides

For in vivo testing of MTHFR inhibitors, the previously described nude mouse cancer model may be used (Bufalo et al., (1996) British J. Canc. 74:387-393; Dean et al., (1996) Cancer Res. 56:3499-3507; Hasegawa et al., (1998) Int. J. Cancer 76:812-816; Narayanan, (1994) In Vivo 8:787-794). Briefly, cancer cells are injected subcutaneously into the flank of a nu/nu athymic mouse. The size of the resulting tumor is measured regularly. When the tumor volume reaches 100 mm³-200 mm³, an MTHFR inhibitor or control compound is injected subcutaneously at the site of the tumor. For initial experiments, a dose of 200 :g inhibitor is injected every other day for a period of one to two weeks. During this period, the appearance and size of the tumor is monitored. After the series of injections is completed, the mouse is sacrificed, and the tumor is removed for further analysis.

After the efficacy of subcutaneous administration of the inhibitor has been determined, intravenous injections may be performed to evaluate the efficacy of various doses and dosing frequencies for systemic administration of the inhibitor. This information may be used to determine the appropriate dosing schedule for human clinical trials. If desired, MTHFR inhibitors may also be tested in other standard animal models for cancer, such as naturally-occurring or induced cancers in other mammals including rats, dogs, or monkeys.

### Example 7

### Effect Of MTHFR Antisense Oligonucleotides on Tumor Growth and MTHFR Protein Levels in vivo

The effect of the methylated antisense oligonucleotide SEQ ID NO:19 was tested in CD1 athymic mice bearing tumors derived from the cancer cell lines SW620 (colon carcinoma) or A549 (lung carcinoma) and compared to mice treated with the control methylated oligonucleotide SEQ ID NO:20.

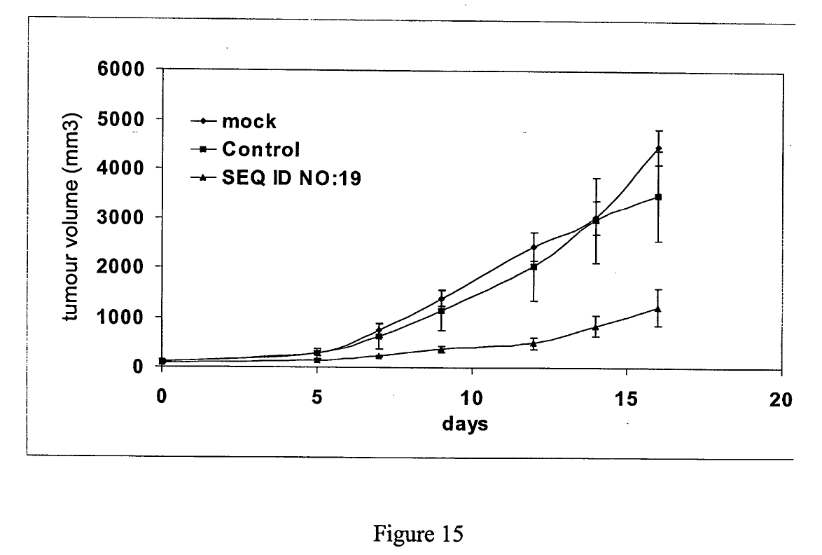

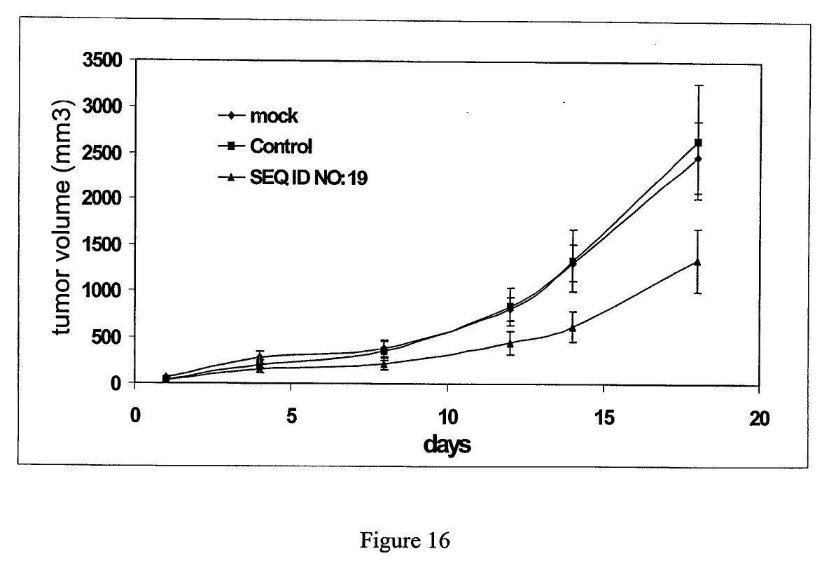

A549 lung tumors or SW620 colon tumors were grafted onto the flank of CD-1 mice. When the tumors reached a volume between 40-150 mm³, the animals were injected intraperitoneally with antisense oligonucleotide SEQ ID NO:19 or control oligonucleotide SEQ ID NO:20 at a dose of 3 mg/kg every day for 14 days. Mock injections were performed with phosphate buffered saline (PBS). Tumor size was monitored by measurement at various timepoints, as indicated in FIGS. 15 and 16.

FIGS. 15 and 16 demonstrate the effect of methylated antisense oligonucleotide SEQ ID NO:19 on the volume of tumors derived from SW620 colon carcinoma cells and A549 lung carcinoma cells, respectively. These results indicate that tumor growth in the group of mice treated with the methylated oligonucleotide was decreased by about 40% in comparison to the group treated with the control oligonucleotide.

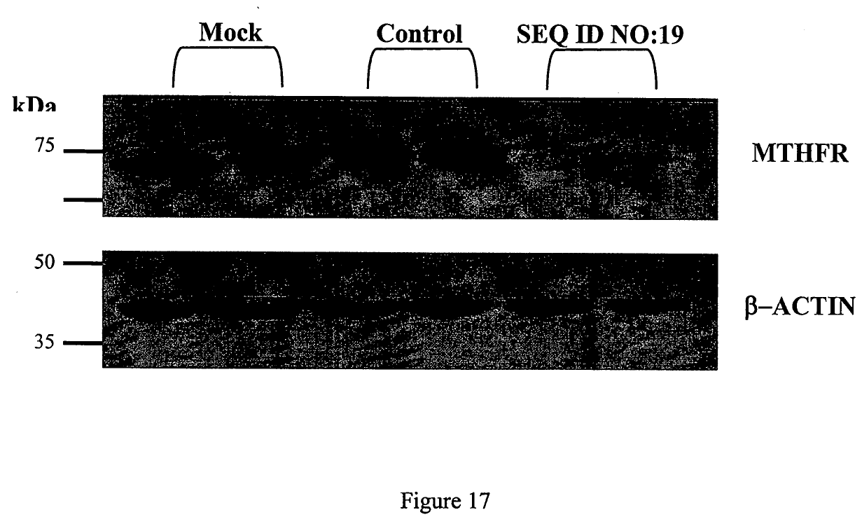

A549 lung tumors from animals treated with oligonucleotides as noted above, were used to prepare protein extracts for Western blotting analysis. Protein extracts were prepared by Polytron treatment of tumors in a lysis buffer followed by centrifugation at 12,000×g; the supernatant was used for Western blotting. Western blotting to quantitate MTHFR protein levels was carried out as described under the “General” methods section. Protein levels of β-actin were also assayed by Western blotting to verify equal loading of sample in each lane. The density of the MTHFR band was quantified relative to the density of the internal control (actin). As shown in FIG. 17, MTHFR protein levels in tumors treated with oligonucleotide SEQ ID NO:19 were 60-70% lower than those treated with the control methylated oligonucleotide.

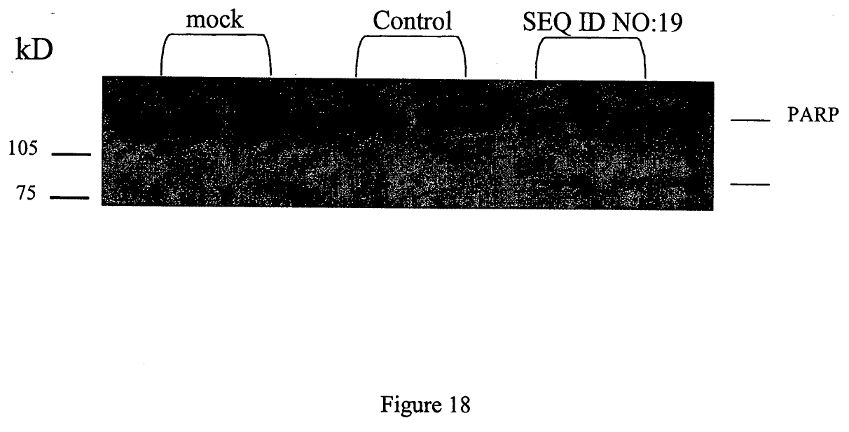

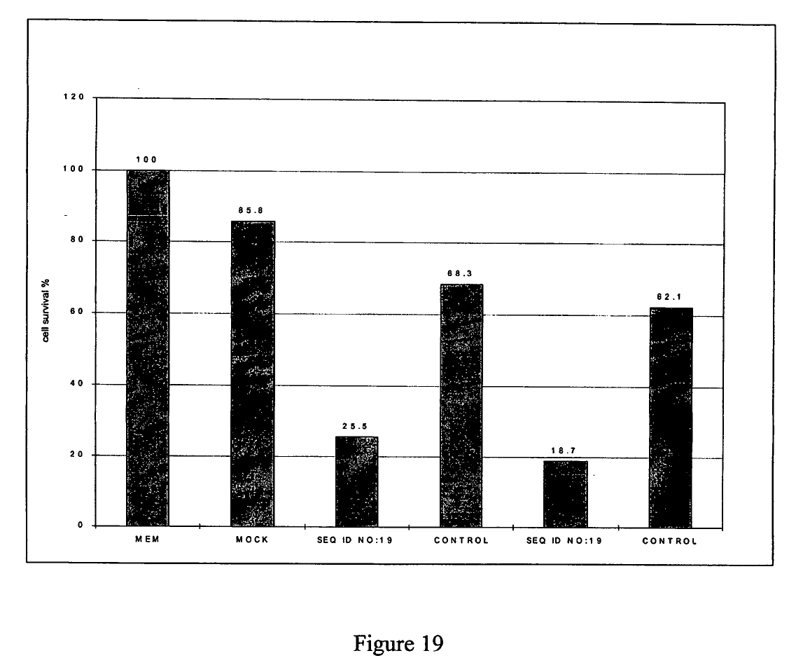

Western blotting was also conducted to quantitate the levels of PARP (poly(ADP-ribose) polymerase) protein as an indicator of apoptosis to determine whether apoptosis was contributing to the decreased size of the tumors treated with the methylated antisense oligonucleotide SEQ ID NO:19. FIG. 18 depicts an example of a PARP Western blot showing both the full length 115 kDa PARP protein and a protein of approximately 85 kDa, which corresponds to a digestion product of PARP. This ˜85 kDa fragment is an marker of apoptosis and indicates that MTHFR inhibition by antisense oligonucleotide SEQ ID NO:19 is associated with increased levels of apoptosis, as demonstrated by the presence of the ˜85 kDa fragment of PARP in the SEQ ID NO:19 lane.

### Example 8

### Effect of MTHFR Antisense Oligonucleotides Alone or in Combination With Anti-Cancer Therapeutics on Growth of Cancer Cell Lines in vitro

The following cancer cell lines were used in these experiments: A549 lung carcinoma ATCC Accession No. CCL-185), SW620 colon carcinoma (ATCC Accession No. CCL-227), SK-N-F1 neuroblastoma (ATCC Accession No. CRL 2142), MCF7 breast cancer (ATCC Accession No. HTB 2), and PC3 prostate cancer (ATCC Accession No. CRL-1435) cell lines.

The cell lines noted above were treated with either methylated antisense oligonucleotide SEQ ID NO:19 or the methylated control oligonucleotide SEQ ID NO:20 alone or in combination with a chemotherapeutic agent. Cells were transfected as described in the “General” methods section, with the following modifications. Cells were transfected with 200 nM antisense oligonucleotide SEQ ID NO:19 or control oligonucleotide SEQ ID NO:20 for 5 hours on 2 consecutive days. Other controls included in the experiment were cells transfected with Lipofectin alone and untransfected cells. The cells were allowed to recover for 2 days before they were counted. When cells were treated with a chemotherapeutic agent in addition to the antisense oligonucleotide, the chemotherapeutic agent was added to the cells after transfection (a period of 5 hours) and incubated with the cells for 18 hours. The concentration of chemotherapeutic agents used is noted below.

FIG. 19 shows the effect of treatment of PC3 prostate carcinoma cells with the methylated oligonucleotide SEQ ID NO:19 alone or the control oligonucleotide SEQ ID NO:20 alone (CONTROL). MOCK refers to cells mock-transfected with Lipofectin and MEM refers to untransfected cells. Treatment of PC3 prostate carcinoma cells with antisense oligonucleotide SEQ ID NO:19 decreased cell numbers compared to the control oligonucleotide.

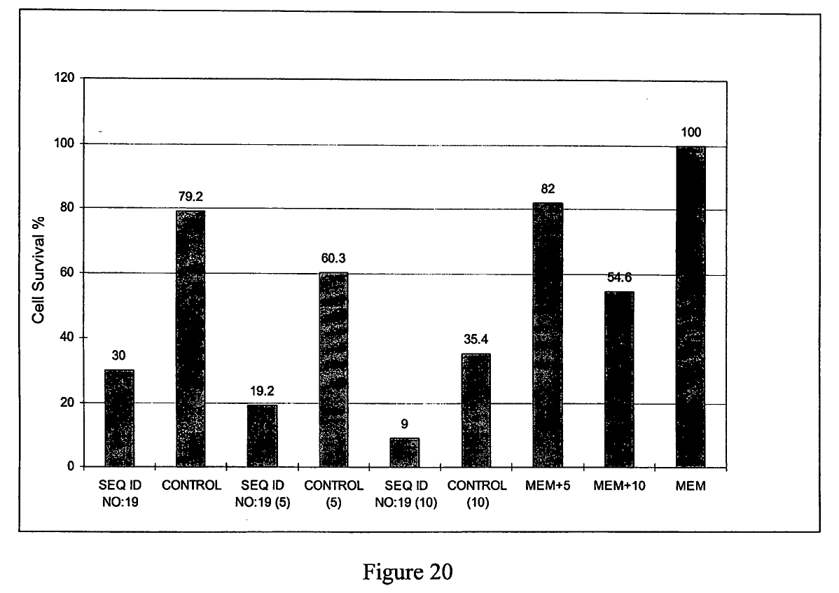

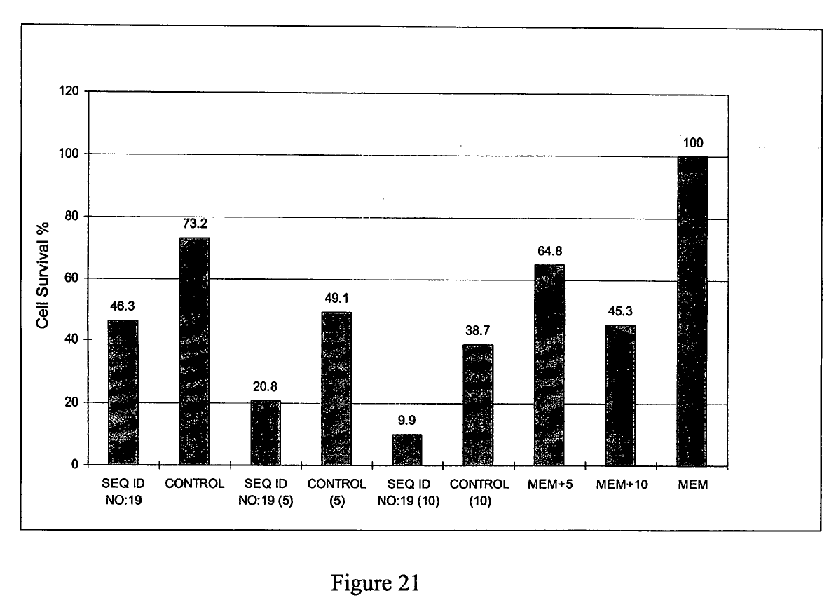

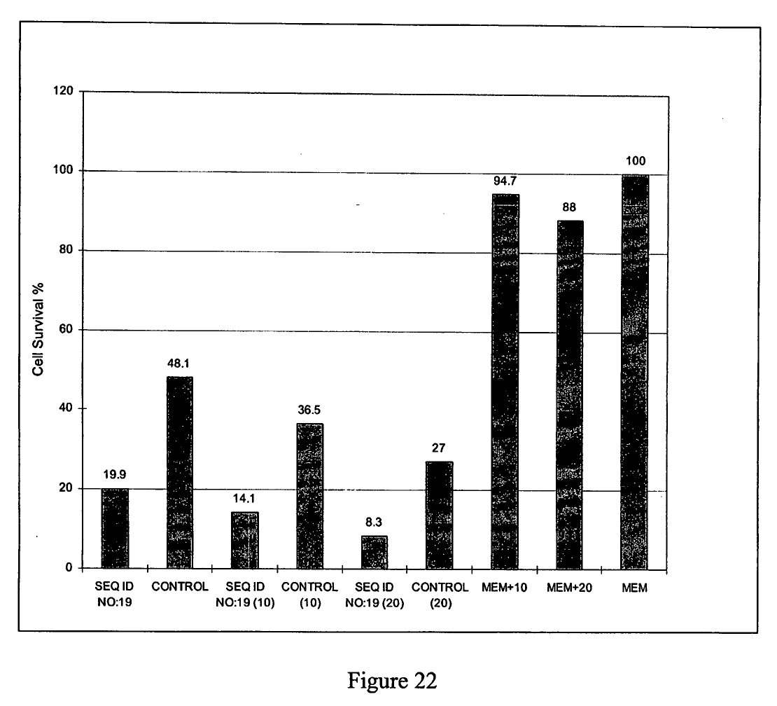

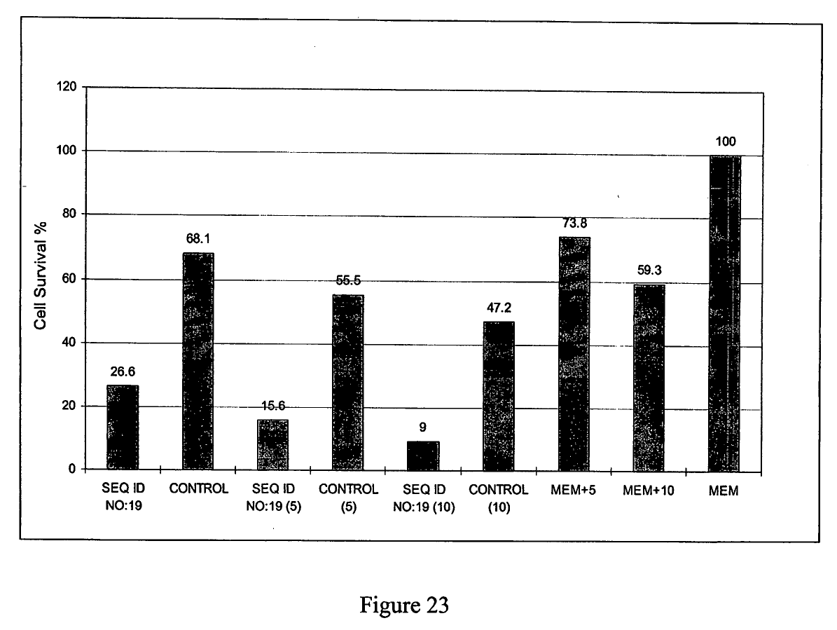

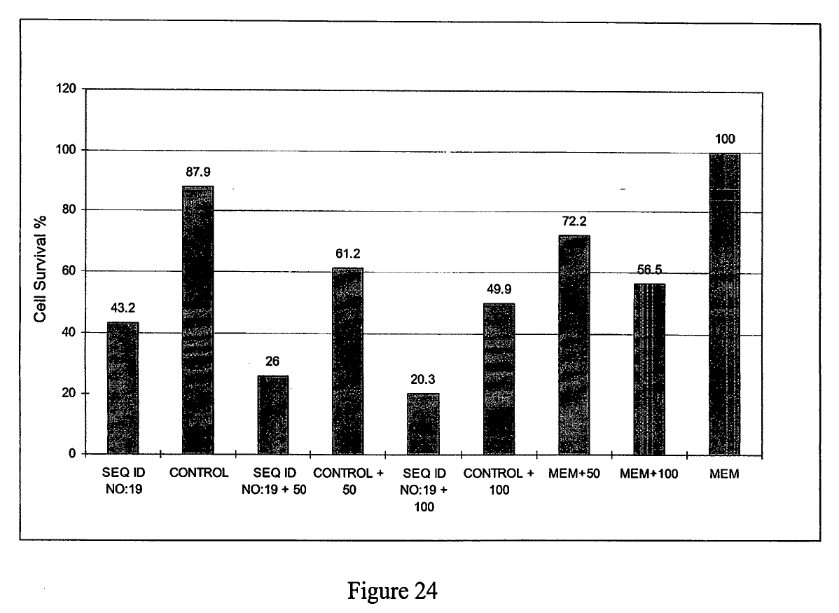

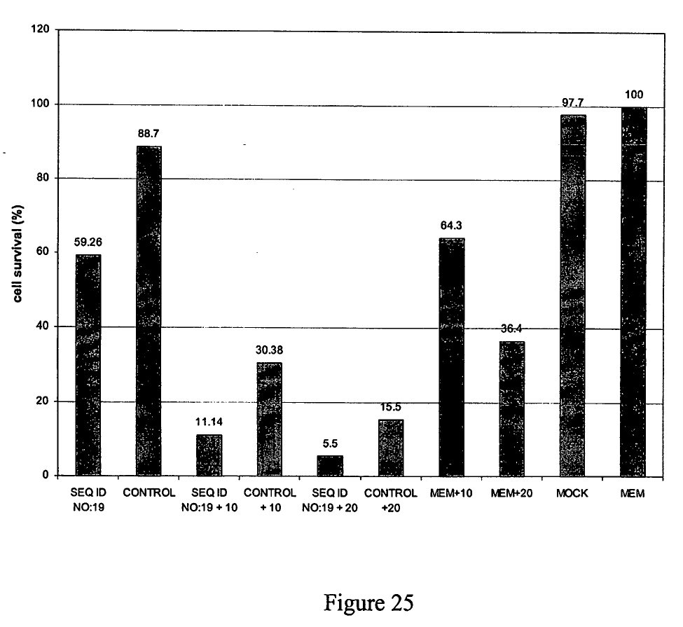

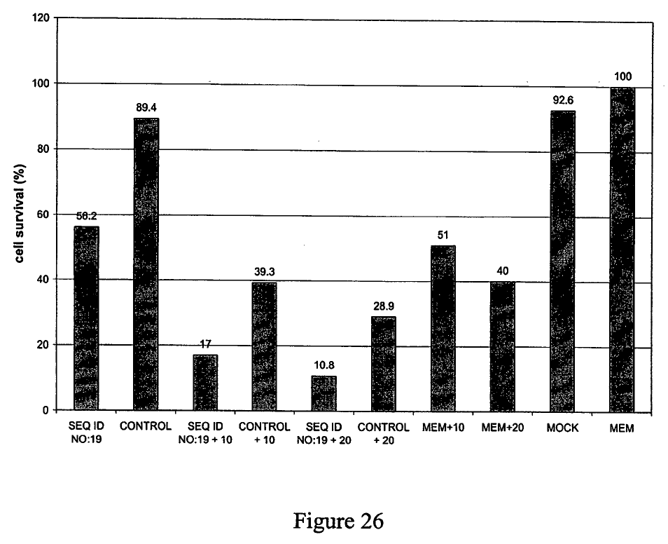

FIGS. 20-26 show the effect of methylated antisense oligonucleotide SEQ ID NO:19 in combination with:
cisplatin (CDDP, 5 μM and 10 μM) on the growth of PC3 prostate carcinoma cells (FIG. 20),
CDDP (5 μM and 10 μM) on the growth of SK-N-F1 neuroblastoma cells (FIG. 21),
CDDP (10 μM and 20 μM) on the growth of MCF-7 breast cancer cells (FIG. 22),
5-FU (5 μM and 10 μM) on the growth of MCF-7 breast cancer cells (FIG. 23),
Taxol® (50 nM and 100 nM) on the growth of MCF-7 breast cancer cells (FIG. 24),
CDDP (10 μM and 20 μM) on the growth of A549 lung carcinoma cells (FIG. 25),
5-FU (10 μM and 20 μM) on the growth of SW620 colon carcinoma cells (FIG. 26).
   CONTROL, MOCK and MEM refer to cells treated with the control oligonucleotide SEQ ID NO:20, cells mock transfected with Lipofectin and untransfected cells, respectively. These results demonstrate that treatment of all cell lines with a combination of the methylated antisense oligonucleotide SEQ ID NO:19 and a chemotherapeutic agent (5-fluorouracil, cisplatin or Taxol®) showed an additive effect. Cell numbers were decreased by 40%-50% compared to therapy with either agent alone.

### Example 9

### Effect of MTHFR Antisense Oligonucleotides in Combination With Anti-Cancer Therapeutics on Growth of Tumors in vivo

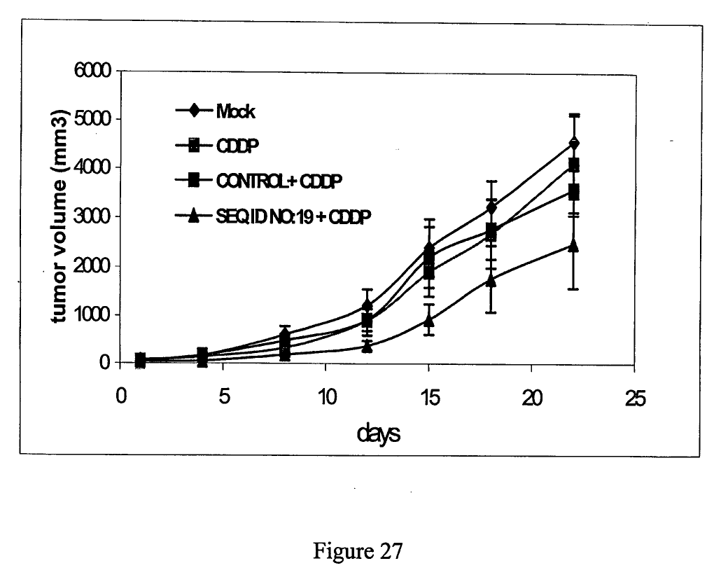

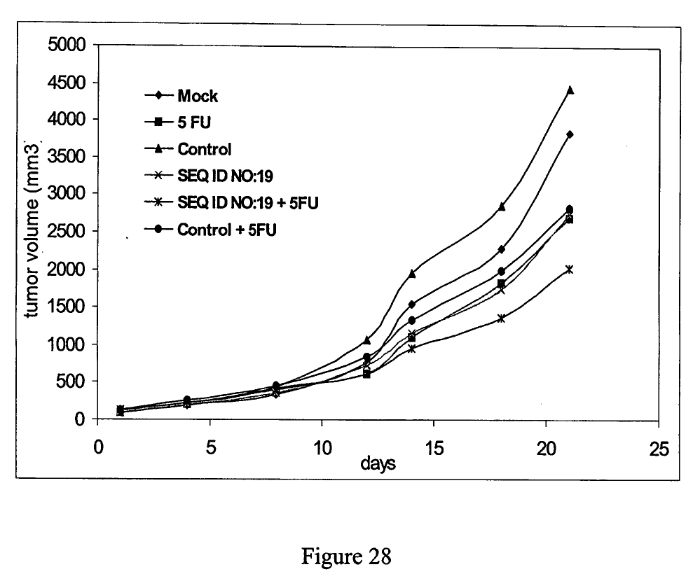

The effect of treatment with the methylated antisense oligonucleotide SEQ ID NO:19 in combination with a chemotherapeutic agent on tumor volume in vivo was investigated in athymic CD-1 nude mice bearing tumors derived from A549 lung carcinoma cells and SW620 colon carcinoma cells. A549 lung tumors or SW620 colon tumors were grafted onto the flank of CD-1 mice. When the tumors reached a volume between 40-150 mm³, the animals were injected intraperitoneally with antisense oligonucleotide SEQ ID NO:19 or control oligonucleotide SEQ ID NO:20 at 3 mg/kg daily, intraperitoneally. Animals grafted with A549 lung tumors were treated with oligonucleotides at 3 mg/kg for 14 days, and were additionally treated with cisplatin at 3 mg/kg injected intraperitoneally once a week for 3 weeks (days 1, 8 and 14). Animals grafted with SW620 tumors were treated with oligonucleotides for 21 days, and were additionally treated with 5-FU at 20 mg/kg injected intraperitoneally once per day for days 4-8 and days 12-16. Measurements of tumor volume were taken at various time points as shown in FIGS. 27 and 28.

The effect of treatment with the methylated antisense oligonucleotide SEQ ID NO:19 alone or in combination with cisplatin (CDDP) on the volume of A549 lung tumors compared to the effect of CDDP alone is shown in FIG. 27. FIG. 28 compares the effect of SEQ ID NO:19 alone, 5-fluorouracil (5-FU) alone and the combination of 5-FU with SEQ ID NO:19 on the volume of SW620 colon tumors. The data presented in these figures show that treatment with the methylated antisense oligonucleotide SEQ ID NO:19 in combination with a chemotherapeutic agent is more effective than treatment with the antisense oligonucleotide or the chemotherapeutic alone. The effect of the combined treatments was additive compared to the effect of the individual treatments.

The disclosure of all patents, publications, including published patent applications, and database entries referenced in this specification are specifically incorporated by reference in their entirety to the same extent as if each such individual patent, publication, and database entry were specifically and individually indicated to be incorporated by reference.

The embodiments of the invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

1. An oligonucleotide inhibitor of methylenetetrahydrofolate reductase (MTHFR) between about 7 and about 100 nucleotides in length comprising a sequence that is complementary to a human MTHFR mRNA, wherein the oligonucleotide inhibits human MTHFR gene expression.

2. The oligonucleotide according to claim 1, wherein the oligonucleotide selectively inhibits cancer cell growth.

3. The oligonucleotide according to claim 1, wherein the oligonucleotide comprises a sequence complementary to exon 5 of the human MTHFR mRNA.

4. The antisense oligonucleotide according to claim 1, wherein the oligonucleotide comprises at least 7 consecutive nucleotides of the sequence as set forth in SEQ ID NO:16 or 19.

5. The oligonucleotide according to claim 1, wherein the oligonucleotide is a single-stranded antisense oligonucleotide.

6. The oligonucleotide according to claim 1, wherein the oligonucleotide is a double-stranded siRNA oligonucleotide.

7. The antisense oligonucleotide according to claim 5, wherein the oligonucleotide is a phosphorothioate nucleic acid.

8. The antisense oligonucleotide according to claim 5, wherein the oligonucleotide comprises one or more methylated cytosine.

9. A vector comprising a nucleic acid encoding the oligonucleotide according to claim 1.

10. A method of treating, stabilizing or preventing cancer in a mammal comprising administering to said mammal an oligonucleotide inhibitor of methylenetetrahydrofolate reductase (MTHFR) between about 7 and about 100 nucleotides in length comprising a sequence that is complementary to a human MTHFR mRNA, wherein the oligonucleotide inhibits human MTHFR gene expression.

11. The method according to claim 10, wherein the oligonucleotide comprises a sequence complementary to exon 5 of human MTHFR mRNA.

12. The method according to claim 10, wherein the oligonucleotide comprises at least 7 consecutive nucleotides of the sequence as set forth in SEQ ID NO:16 or 19.

13. The method according to claim 10, wherein the oligonucleotide is a single-stranded antisense oligonucleotide.

14. The method according to claim 10, wherein the oligonucleotide is a double-stranded siRNA oligonucleotide.

15. The method according to claim 10, wherein said mammal is a human.

16. The method according to claim 10, wherein said cancer is a solid tumor.

17. The method according to claim 10, wherein said cancer is a carcinoma.

18. The method according to claim 10, wherein said cancer is an adenocarcinoma.

19. The method according to claim 10, wherein said cancer is breast cancer, colon cancer, colorectal cancer, lung cancer, prostate cancer, cancer of the nervous system or brain cancer.

20. A method of treating, stabilizing or preventing cancer in a mammal comprising administering to said mammal an oligonucleotide inhibitor of methylenetetrahydrofolate reductase (MTHFR) in combination with one or more chemotherapeutic, wherein the oligonucleotide is between about 7 and about 100 nucleotides in length comprising a sequence that is complementary to a human MTHFR mRNA, and wherein the oligonucleotide inhibits human MTHFR gene expression.

21. The method according to claim 20, wherein oligonucleotide comprises a sequence complementary to exon 5 of the human MTHFR mRNA.

22. The method according to claim 20, wherein the oligonucleotide comprises at least 7 consecutive nucleotides of the sequence as set forth in SEQ ID NO:16 or 19.

23. The method according to claim 20, wherein the oligonucleotide is a single-stranded antisense oligonucleotide.

24. The method according to claim 20, wherein the oligonucleotide is a double-stranded siRNA oligonucleotide.

25. The method according to claim 20, wherein said mammal is a human.

26. The method according to claim 20, wherein said cancer is a solid tumor.

27. The method according to claim 20, wherein said cancer is a carcinoma.

28. The method according to claim 20, wherein said cancer is an adenocarcinoma.

29. The method according to claim 20, wherein said cancer is breast cancer, colon cancer, colorectal cancer, lung cancer, prostate cancer, cancer of the nervous system or brain cancer.

30. The method according to claim 20, wherein said chemotherapeutic is cisplatin, 5-fluorouracil or taxol, or a combination thereof.

31. A method of inhibiting growth of cancer cells comprising the step of contacting said cancer cells with an oligonucleotide inhibitor of methylenetetrahydrofolate reductase (MTHFR) between about 7 and about 100 nucleotides in length comprising a sequence that is complementary to a human MTHFR mRNA, wherein the oligonucleotide inhibits human MTHFR gene expression.
